SEQUENCE LISTING (53 sequences)

SEQ ID NO: 1 - protein, 11 aa, Homo sapiens
  MISC_FEATURE (1)..(1): Xaa is either absent or selected from the group consisting of YLRIVQ, LRIVQ, RIVQ, IVQ, VQ, and Q
  MISC_FEATURE (11)..(11): Xaa is either absent or Phe
XCRSVEGSCGX

SEQ ID NO: 2 - protein, 16 aa, Homo sapiens
YLRIVQCRSVEGSCGF

SEQ ID NO: 3 - protein, 15 aa, Homo sapiens
LRIVQCRSVEGSCGF

SEQ ID NO: 4 - protein, 9 aa, Homo sapiens
CRSVEGSCG

SEQ ID NO: 5 - protein, 10 aa, Homo sapiens
CRSVEGSCGF

SEQ ID NO: 6 - protein, 11 aa, Homo sapiens
  MISC_FEATURE (1)..(1): Xaa is absent or selected from the group consisting of YLRVMK, LRVMK, RVMK, VMK, MK, and K
  MISC_FEATURE (11)..(11): Xaa is absent or selected from the group consisting of A and AF
XCRRFVESSCX

SEQ ID NO: 7 - protein, 17 aa, Homo sapiens
YLRVMKCRRFVESSCAF

SEQ ID NO: 8 - protein, 16 aa, Homo sapiens
LRVMKCRRFVESSCAF

SEQ ID NO: 9 - protein, 11 aa, Homo sapiens
CRRFVESSCAF

SEQ ID NO: 10 - protein, 10 aa, Homo sapiens
CRRFVESSCA

SEQ ID NO: 11 - protein, 12 aa, Homo sapiens
  MISC_FEATURE (1)..(1): Xaa is absent or selected from the group consisting of S, HS, GHS, PGHS, APGHS, EAPGHS, SEAPGHS, SSEAPGHS, PSSEAPGHS, DPSSEAPGHS, and IDPSSEAPGHS
  MISC_FEATURE (4)..(4): Xaa is selected from the group consisting of Ser, Ala, Val, Leu, Iso and Gly
  MISC_FEATURE (5)..(5): Xaa is selected from the group consisting of Arg and Lys
  MISC_FEATURE (7)..(7): Xaa is selected from the group consisting of Ser, Ala, Val, Leu, Iso and Gly
  MISC_FEATURE (8)..(8): Xaa is selected from the group consisting of Glu and Asp
  MISC_FEATURE (9)..(10): Xaa is selected from the group consisting of Ser, Ala, Val, Leu, Iso and Gly
  MISC_FEATURE (12)..(12): Xaa is absent or selected from the group consisting of S, SS, SSK, SSKF, SSKFS, SSKFSW, SSKFSWD, SSKFSWDE, SSKFSWDEY, SSKFSWDEYE, SSKFSWDEYEQ, SSKFSWDEYEQY, SSKFSWDEYEQYK, SSKFSWDEYEQYKK, and SSKFSWDEYEQYKKE
XCRXXPXXXXCX

SEQ ID NO: 12 - protein, 2 aa, Homo sapiens
HS

SEQ ID NO: 13 - protein, 3 aa, Homo sapiens
GHS

SEQ ID NO: 14 - protein, 4 aa, Homo sapiens
PGHS

SEQ ID NO: 15 - protein, 5 aa, Homo sapiens
APGHS

SEQ ID NO: 16 - protein, 6 aa, Homo sapiens
EAPGHS

SEQ ID NO: 17 - protein, 7 aa, Homo sapiens
SEAPGHS

SEQ ID NO: 18 - protein, 8 aa, Homo sapiens
SSEAPGHS

SEQ ID NO: 19 - protein, 9 aa, Homo sapiens
PSSEAPGHS

SEQ ID NO: 20 - protein, 10 aa, Homo sapiens
DPSSEAPGHS

SEQ ID NO: 21 - protein, 11 aa, Homo sapiens
IDPSSEAPGHS

SEQ ID NO: 22 - protein, 2 aa, Homo sapiens
SS

SEQ ID NO: 23 - protein, 3 aa, Homo sapiens
SSK

SEQ ID NO: 24 - protein, 4 aa, Homo sapiens
SSKF

SEQ ID NO: 25 - protein, 6 aa, Homo sapiens
CSSKFS

SEQ ID NO: 26 - protein, 6 aa, Homo sapiens
SSKFSW

SEQ ID NO: 27 - protein, 7 aa, Homo sapiens
SSKFSWD

SEQ ID NO: 28 - protein, 8 aa, Homo sapiens
SSKFSWDE

SEQ ID NO: 29 - protein, 9 aa, Homo sapiens
SSKFSWDEY

SEQ ID NO: 30 - protein, 10 aa, Homo sapiens
SSKFSWDEYE

SEQ ID NO: 31 - protein, 11 aa, Homo sapiens
SSKFSWDEYEQ

SEQ ID NO: 32 - protein, 12 aa, Homo sapiens
SSKFSWDEYEQY

SEQ ID NO: 33 - protein, 13 aa, Homo sapiens
SSKFSWDEYEQYK

SEQ ID NO: 34 - protein, 14 aa, Homo sapiens
SSKFSWDEYEQYKK

SEQ ID NO: 35 - protein, 15 aa, Homo sapiens
SSKFSWDEYEQYKKE

SEQ ID NO: 36 - protein, 11 aa, Homo sapiens
SCRSRPVESSC

SEQ ID NO: 37 - protein, 10 aa, Homo sapiens
CRSRPVESSC

SEQ ID NO: 38 - protein, 11 aa, Homo sapiens
CRSRPVESSCS

SEQ ID NO: 39 - protein, 12 aa, Homo sapiens
SCRSRPVESSCS

SEQ ID NO: 40 - protein, 11 aa, Homo sapiens
  MISC_FEATURE (1)..(1): Xaa is absent or selected from the group consisting of YLKLLK, LKLLK, KLLK, LLK, LL and K
  MISC_FEATURE (5)..(5): Xaa is selected from the group consisting of Iso and Val
  MISC_FEATURE (6)..(6): Xaa is selected from the group consisting of His and Tyr
  MISC_FEATURE (7)..(7): Xaa is selected from the group consisting of Asp and Asn
  MISC_FEATURE (8)..(8): Xaa is selected from the group consisting of Asn and Ser
  MISC_FEATURE (11)..(11): Xaa is absent or selected from the group consisting of Gly, and a pharmaceutically acceptable carrier
XCRIXXXXNCX

SEQ ID NO: 41 - protein, 9 aa, Homo sapiens
CRIIHNNNC

SEQ ID NO: 42 - protein, 10 aa, Homo sapiens
CRIIHNNNCG

SEQ ID NO: 43 - protein, 9 aa, Homo sapiens
CRIVYDSNC

SEQ ID NO: 44 - protein, 10 aa, Homo sapiens
CRIVYDSNCG

SEQ ID NO: 45 - protein, 6 aa, Homo sapiens
YLRIVQ

SEQ ID NO: 46 - protein, 5 aa, Homo sapiens
LRIVQ

SEQ ID NO: 47 - protein, 4 aa, Homo sapiens
RIVQ

SEQ ID NO: 48 - protein, 6 aa, Homo sapiens
YLRVMK

SEQ ID NO: 49 - protein, 5 aa, Homo sapiens
LRVMK

SEQ ID NO: 50 - protein, 4 aa, Homo sapiens
RVMK

SEQ ID NO: 51 - protein, 6 aa, Homo sapiens
YLKLLK

SEQ ID NO: 52 - protein, 5 aa, Homo sapiens
LKLLK

SEQ ID NO: 53 - protein, 4 aa, Homo sapiens
KLLK

(19) United States
(12) Patent Application Publication
(10) Pub. No.: US 2025/0255934 A1
(43) Pub. Date: Aug. 14, 2025

(54) COMPOSITIONS FOR TREATING INFLAMMATORY AIRWAY DISEASE AND USES THEREOF

(71) Applicant: Lateral IP Pty Ltd, Melbourne, Victoria (AU)

(72) Inventors: Andrew GEARING, Melbourne, Victoria (AU); David KENLEY, Melbourne, Victoria (AU)

(73) Assignee: Lateral IP Pty Ltd, Melbourne, Victoria (AU)

(21) Appl. No.: 18/559,118

(22) PCT Filed: May 6, 2022
(86) PCT No.: PCT/AU2022/050427

§ 371 Date: Nov. 6, 2023

(30) Foreign Application Priority Data
May 7, 2021 (AU) 2021901369

(51) Int. Cl. A61K 38/22; A61P 11/00

(52) U.S. Cl. CPC A61K 38/2257; A61P 11/00

(57) ABSTRACT

The present disclosure relates to the use of C-terminal fragments of human and non-human growth hormone, synthetic cyclic peptides and C-terminal fragments of human prolactin precursor for the treatment of inflammatory airway disease in a subject in need thereof.

### CROSS-REFERENCE TO RELATED APPLICATIONS

The present application is the U.S. National Stage of International Application No. PCT/AU2022/050427, filed May 6, 2022, and claims priority to Australian Patent Application No. 2021901369 filed May 7, 2021.

### SEQUENCE LISTING

The instant application contains a Sequence Listing which has been submitted electronically in ASCII format and is hereby incorporated by reference in its entirety. Said ASCII file, created on Jun. 2, 2024, is named “017227-0265_SL.txt” and is 10.2 kilobytes in size.

### FIELD OF THE INVENTION

The invention relates generally to peptides suitable for treating an inflammatory airway disease and uses thereof.

### BACKGROUND

All references, including any patent or patent application cited in this specification are hereby incorporated by reference to enable full understanding of the invention. Nevertheless, such references are not to be read as constituting an admission that any of these documents forms part of the common general knowledge in the art, in Australia or in any other country.

Inflammatory airway diseases, such as chronic obstructive pulmonary disease (COPD), asthma, chronic bronchitis, emphysema, cystic fibrosis, lung cancer and bronchopulmonary dysplasia, are among the world's most prevalent diseases. The prevalence of asthma, in particular, has increased over the past 20 years and currently affects up to 10% of the populations in most developed countries. COPD is the sixth most common cause of death in the world and is said to affect around 4-6% of people of 45 years of age or more. It is beyond contestation that inflammatory airway diseases constitute a major financial burden to society, having regard to both direct and indirect costs.

Asthma and COPD are identified by the presence of characteristic symptoms and functional abnormalities, with airway obstruction being the sine qua non of both diseases. The airway obstruction in asthma is typically reversible, whereas COPD is typically characterized by abnormal expiratory flow that does not change markedly over periods of several months of observation. Both airway diseases are associated with lung inflammation induced by different initiating factors, examples of which include environmental allergens and carcinogens, occupational sensitizing agents, cigarette smoke, asbestos and silica. It is to be noted, however, that some patients with asthma who do not smoke will also develop irreversible airway obstruction similar to COPD.

Chronic obstructive pulmonary disease is a growing healthcare problem that is expected to worsen as the population ages and the worldwide use of tobacco products increases. Smoking cessation is the only effective means of prevention. Employers are in a unique position to help employees stop smoking. During the long asymptomatic phase, lung function nevertheless continues to decline; therefore, many patients seek medical attention only when they are at an advanced stage or when they have experienced an acute exacerbation. To help preserve patients' quality of life and reduce healthcare costs related to this chronic disease, clinicians need to accurately diagnose the condition and appropriately manage patients through the long course of their illness. This article discusses the current approach to patient management.

As noted by Devine, FJ (2008; Am Health Drug Benefits; 1(7):34-42), COPD is a poorly reversible disease of the lungs that is one of the major causes of morbidity and mortality worldwide. Contrary to the trends for other major chronic diseases in the United States, the prevalence of and mortality from COPD have continued to rise, with death rates having doubled between 1970 and 2002, and mortality figures for women having now surpassed those for men. Given that the majority of COPD cases are caused by smoking, it is primarily a preventable disease. Most patients with COPD are middle-aged or elderly. Effective treatments for COPD have largely been elusive. The only strategy known to reduce the incidence of the disease is smoking cessation.

Asthma is a heterogeneous, multifactorial disease with variable and mostly reversible respiratory pathway obstruction based on a chronic bronchial inflammatory reaction (Horak et al., 2016; Wien Klin Wochenschr. 128(15):541-554). Symptoms of asthma (cough, phlegm, rhonchus, wheezing, chest tightness, or shortness of breath) are variable and typically correlated with expiratory flow limitation. Owing to its heterogeneity, a number of different phenotypes can be ascribed to asthma and include: allergic asthma, non-allergic asthma, pediatric asthma/recurrent obstructive bronchitis, late-onset asthma, asthma with fixed airflow obstruction, obesity-related asthma, occupational asthma, asthma in the elderly and severe asthma.

Treatment for asthma (pharmacological and non-pharmacological intervention) is largely based on symptom control—a cycle of assess, adjust, and review—and is usually associated with reduced asthma exacerbations. From a pharmacological perspective, the gold standard in asthma therapy is typically low-dose inhaled corticosteroids, often in combination with an on-demand short-acting beta-2-agonist (SABA). Other treatments include LTRA (leucotriene-receptor antagonists), combinations of low-dose inhaled corticosteroids and long-acting beta-2-agonist (LABA). However, existing treatments have the potential to cause side effects, in particular during long-term use. Common side effects of preventative medication (e.g., inhaled corticosteroids) are a hoarse voice, sore mouth and throat, and fungal infections of the throat.

Hence, while there have been some advances in the treatment of inflammatory airway diseases, such as COPD and asthma, there remains an urgent need for broad-spectrum treatment strategies effective for alleviating inflammatory airway diseases with limited or minimal side effects. The present invention solves, or at least partly alleviates this problem by providing compositions that are effective at treating inflammatory airway diseases, including COPD.

### SUMMARY OF THE INVENTION

In an aspect disclosed herein, there is provided a method of treating an inflammatory airway disease in a subject, the method comprising administering to a subject in need thereof a therapeutically effective amount of a peptide of formula (I), or a pharmaceutically acceptable salt thereof:

(I)
(SEQ ID NO: 1)
R¹-CRSVEGSCG-R²

wherein
R¹ is selected from the group consisting of YLRIVQ (SEQ ID NO:45), LRIVQ (SEQ ID NO:46), RIVQ (SEQ ID NO:47), IVQ, VQ, and Q, or R¹ is absent; and
R² is F (phenylalanine), or R² is absent.

In an embodiment, the peptide is selected from the group consisting of

(SEQ ID NO: 2)
YLRIVQCRSVEGSCGF,
(SEQ ID NO: 3)
LRIVQCRSVEGSCGF,
(SEQ ID NO: 4)
CRSVEGSCG
and
(SEQ ID NO: 5)
CRSVEGSCGF.

In another aspect disclosed herein, there is provided a pharmaceutical composition comprising a peptide of formula (I), or a pharmaceutically acceptable salt thereof, for use in the treatment of an inflammatory airway disease in a subject:

(I)
(SEQ ID NO: 1)
R¹-CRSVEGSCG-R²

wherein
R¹ is selected from the group consisting of YLRIVQ (SEQ ID NO:45), LRIVQ (SEQ ID NO:46), RIVQ (SEQ ID NO:47), IVQ, VQ, and Q, or R¹ is absent; and
R² is F (phenylalanine), or R² is absent.

In another aspect disclosed herein, there is provided use of a peptide of formula (I), or a pharmaceutically acceptable salt thereof, in the manufacture of a medicament for the treatment of an inflammatory airway disease in a subject:

(I)
(SEQ ID NO: 1)
R¹-CRSVEGSCG-R²

wherein
R¹ is selected from the group consisting of YLRIVQ (SEQ ID NO:45), LRIVQ (SEQ ID NO:46), RIVQ (SEQ ID NO:47), IVQ, VQ, and Q, or R¹ is absent; and
R² is F (phenylalanine), or R² is absent.

In another aspect disclosed herein, there is provided a method of treating an inflammatory airway disease in a subject, the method comprising administering to a subject a therapeutically effective amount of a peptide of formula (II), or a pharmaceutically acceptable salt thereof:

(II)
(SEQ ID NO: 6)
R¹-CRRFVESSC-R²

wherein
R¹ is selected from the group consisting of YLRVMK (SEQ ID NO:48), LRVMK (SEQ ID NO:49), RVMK (SEQ ID NO:50), VMK, MK, and K, or R¹ is absent; and
R² is selected from the group consisting of A (alanine) and AF (alanine-phenylalanine), or R² is absent.

In another aspect disclosed herein, there is provided a pharmaceutical composition comprising a peptide of formula (II), or a pharmaceutically acceptable salt thereof, for use in the treatment of an inflammatory airway disease in a subject:

(II)
(SEQ ID NO: 6)
R¹-CRRFVESSC-R²

wherein
R¹ is selected from the group consisting of YLRVMK (SEQ ID NO:48), LRVMK (SEQ ID NO:49), RVMK (SEQ ID NO:50), VMK, MK, and K, or R¹ is absent; and R² is selected from the group consisting of A (alanine) and AF (alanine-phenylalanine), or R² is absent.

In another aspect disclosed herein, there is provided a use of a peptide of formula (II), or a pharmaceutically acceptable salt thereof, in the manufacture of a medicament for the treatment of an inflammatory airway disease in a subject:

(II)
(SEQ ID NO: 6)
R¹-CRRFVESSC-R²

wherein
R¹ is selected from the group consisting of YLRVMK (SEQ ID NO:48), LRVMK (SEQ ID NO:49), RVMK (SEQ ID NO:50), VMK, MK, and K, or R¹ is absent; and
R² is selected from the group consisting of A (alanine) and AF (alanine-phenylalanine), or R² is absent.

In an embodiment, the peptide is selected from the group consisting of

(SEQ ID NO: 7)
YLRVMKCRRFVESSCAF,
(SEQ ID NO: 8)
LRVMKCRRFVESSCAF,
(SEQ ID NO: 9)
CRRFVESSCAF
and
(SEQ ID NO: 10)
CRRFVESSCA.

In another aspect disclosed herein, there is provided a method of treating an inflammatory airway disease in a subject, the method comprising administering to a subject in need thereof a therapeutically effective amount of a peptide of formula (III), or a pharmaceutically acceptable salt thereof:

(III)
(SEQ ID NO: 11)
R¹-C-R-X¹-X²-P-X³-X⁴-X⁵-X⁶-C-R²

wherein
X¹, X³, X⁵, and X⁶ is an amino acid residue selected from the group consisting of serine, alanine, valine, leucine, isoleucine and glycine;
X² is arginine or lysine;
X⁴ is glutamic acid or aspartic acid;
R¹ is selected from the group consisting of:
S,

(SEQ ID NO: 12)
HS,
(SEQ ID NO: 13)
GHS,
(SEQ ID NO: 14)
PGHS,
(SEQ ID NO: 15)
APGHS,
(SEQ ID NO: 16)
EAPGHS,
(SEQ ID NO: 17)
SEAPGHS,
(SEQ ID NO: 18)
SSEAPGHS,
(SEQ ID NO: 19)
PSSEAPGHS,
(SEQ ID NO: 20)
DPSSEAPGHS
and
(SEQ ID NO: 21)
IDPSSEAPGHS,

or R¹ is absent; and
R² is selected from the group consisting of
S,

(SEQ ID NO: 22)
SS,
(SEQ ID NO: 23)
SSK,
(SEQ ID NO: 24)
SSKF,
(SEQ ID NO: 25)
SSKFS,
(SEQ ID NO: 26)
SSKFSW,
(SEQ ID NO: 27)
SSKFSWD,
(SEQ ID NO: 28)
SSKFSWDE,
(SEQ ID NO: 29)
SSKFSWDEY,
(SEQ ID NO: 30)
SSKFSWDEYE,
(SEQ ID NO: 31)
SSKFSWDEYEQ,
(SEQ ID NO: 32)
SSKFSWDEYEQY,
(SEQ ID NO: 33)
SSKFSWDEYEQYK,
(SEQ ID NO: 34)
SSKFSWDEYEQYKK,
and
(SEQ ID NO: 35)
SSKFSWDEYEQYKKE,

or R² is absent;
or a pharmaceutically acceptable salt thereof.

In another aspect disclosed herein, there is provided a pharmaceutical composition comprising a peptide of formula (III), or a pharmaceutically acceptable salt thereof, for use in the treatment of an inflammatory airway disease in a subject:

(III)
(SEQ ID NO: 11)
R¹-C-R-X¹-X²-P-X³-X⁴-X⁵-X⁶-C-R²

wherein
X¹, X³, X⁵, and X⁶ is an amino acid residue selected from the group consisting of serine, alanine, valine, leucine, isoleucine and glycine;
X² is arginine or lysine;
X⁴ is glutamic acid or aspartic acid;
R¹ is selected from the group consisting of:
S,

(SEQ ID NO: 12)
HS,
(SEQ ID NO: 13)
GHS,
(SEQ ID NO: 14)
PGHS,
(SEQ ID NO: 15)
APGHS,
(SEQ ID NO: 16)
EAPGHS,
(SEQ ID NO: 17)
SEAPGHS,
(SEQ ID NO: 18)
SSEAPGHS,
(SEQ ID NO: 19)
PSSEAPGHS,
(SEQ ID NO: 20)
DPSSEAPGHS
and
(SEQ ID NO: 21)
IDPSSEAPGHS,

or R¹ is absent; and
R² is selected from the group consisting of
S,

(SEQ ID NO: 22)
SS,
(SEQ ID NO: 23)
SSK,
(SEQ ID NO: 24)
SSKF,
(SEQ ID NO: 25)
SSKFS,
(SEQ ID NO: 26)
SSKFSW,
(SEQ ID NO: 27)
SSKFSWD,
(SEQ ID NO: 28)
SSKFSWDE,
(SEQ ID NO: 29)
SSKFSWDEY,
(SEQ ID NO: 30)
SSKFSWDEYE,
(SEQ ID NO: 31)
SSKFSWDEYEQ,
(SEQ ID NO: 32)
SSKFSWDEYEQY,
(SEQ ID NO: 33)
SSKFSWDEYEQYK,
(SEQ ID NO: 34)
SSKFSWDEYEQYKK,
and
(SEQ ID NO: 35)
SSKFSWDEYEQYKKE,

or R² is absent.

In another aspect disclosed herein, there is provided a use of a peptide of formula (III), or a pharmaceutically acceptable salt thereof, in the manufacture of a medicament for the treatment of an inflammatory airway disease in a subject:

(III)
(SEQ ID NO: 11)
R¹-C-R-X¹-X²-P-X³-X⁴-X⁵-X⁶-C-R²

wherein
X¹, X³, X⁵, and X⁶ is an amino acid residue selected from the group consisting of serine, alanine, valine, leucine, isoleucine and glycine;
X² is arginine or lysine;
X⁴ is glutamic acid or aspartic acid;
R¹ is selected from the group consisting of:
S,

(SEQ ID NO: 12)
HS,
(SEQ ID NO: 13)
GHS,
(SEQ ID NO: 14)
PGHS,
(SEQ ID NO: 15)
APGHS,
(SEQ ID NO: 16)
EAPGHS,
(SEQ ID NO: 17)
SEAPGHS,
(SEQ ID NO: 18)
SSEAPGHS,
(SEQ ID NO: 19)
PSSEAPGHS,
(SEQ ID NO: 20)
DPSSEAPGHS
and
(SEQ ID NO: 21)
IDPSSEAPGHS,

or R¹ is absent; and
R² is selected from the group consisting of
S,

(SEQ ID NO: 22)
SS,
(SEQ ID NO: 23)
SSK,
(SEQ ID NO: 24)
SSKF,
(SEQ ID NO: 25)
SSKFS,
(SEQ ID NO: 26)
SSKFSW,
(SEQ ID NO: 27)
SSKFSWD,
(SEQ ID NO: 28)
SSKFSWDE,
(SEQ ID NO: 29)
SSKFSWDEY,
(SEQ ID NO: 30)
SSKFSWDEYE,
(SEQ ID NO: 31)
SSKFSWDEYEQ,
(SEQ ID NO: 32)
SSKFSWDEYEQY,
(SEQ ID NO: 33)
SSKFSWDEYEQYK,
(SEQ ID NO: 34)
SSKFSWDEYEQYKK,
and
(SEQ ID NO: 35)
SSKFSWDEYEQYKKE,

or R² is absent.

In another aspect disclosed herein, there is provided a method of treating an inflammatory airway disease in a subject, the method comprising administering to a subject in need thereof a therapeutically effective amount of a peptide of formula (IV) or a pharmaceutically acceptable salt thereof:

(IV)
(SEQ ID NO: 40)
R¹-C-R-I-X₁-X₂-X₃-X₄-N-C-R²

wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK (SEQ ID NO:51), LKLLK (SEQ ID NO:52), KLLK (SEQ ID NO:53, LLK, LK, K or R¹ is absent; and
R² is G (glycine), or R² is absent, or R² is a pharmaceutically acceptable carrier.

In another aspect disclosed herein, there is provided a pharmaceutical composition comprising a peptide of formula (IV) or a pharmaceutically acceptable salt thereof, for use in the treatment of an inflammatory airway disease in a subject:

(IV)
(SEQ ID NO: 40)
R¹-C-R-I-X¹-X²-X³-X⁴-N-C-R²

wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK (SEQ ID NO:51), LKLLK (SEQ ID NO:52), KLLK (SEQ ID NO:53, LLK, LK, K or R¹ is absent; and
R² is G (glycine), or R² is absent, or R² is a pharmaceutically acceptable carrier.

In another aspect disclosed herein, there is provided a use of a peptide of formula (IV) or a pharmaceutically acceptable salt thereof, in the manufacture of a medicament for the treatment of an inflammatory airway disease in a subject:

(IV)
(SEQ ID NO: 40)
R¹-C-R-I-X¹-X²-X³-X⁴-N-C-R²

wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK (SEQ ID NO:51), LKLLK (SEQ ID NO:52), KLLK (SEQ ID NO:53, LLK, LK, K or R¹ is absent; and
R² is G (glycine), or R² is absent, or R² is a pharmaceutically acceptable carrier.

In an embodiment, the peptide of formula (IV) is selected from the group consisting of amino acid sequence CRIIHNNNC (SEQ ID NO:41), CRIIHNNNCG (SEQ ID NO:42), CRIVYDSNC (SEQ ID NO:43) and CRIVYDSNCG (SEQ ID NO:44).

### BRIEF DESCRIPTION OF THE FIGURES

FIG. 1 shows that cigarette smoke (CS)-induced airway inflammation was alleviated with LAT8881 treatment in an experimental COPD model. (A) Total cells (B) Macrophages (C) Neutrophils (D) Lymphocytes in the bronchoalveolar lavage fluid (BALF). Data are expressed as mean+/−SEM, and analysed using One-Way ANOVA, n=7-8,*P<0.05, **P<0.01, ***P<0.001 and Mann-Whitney test, n=7-8, ###P<0.001.

FIG. 2 shows that lung function is rescued with LAT8881 treatment in an experimental COPD model. (A) Forced vital capacity (FVC); (B) Total lung capacity; (C) Airway resistance; (D) Inspiratory capacity; (E) Transpulmonary resistance; (F) Peak expiration flow (PEF) rate; (G) Total lung compliance (mL·cmH₂O); (H) Tissue damping; (I) Dynamic elastance; (J) Tissue elastance; (K) Dynamic compliance. Data are expressed as mean+/−SEM, and analysed using One-Way ANOVA, n=8,*P<0.05, **P<0.01 and unpaired students t-test, n=8, #P<0.05, ##P<0.01.

FIG. 3 shows that cigarette smoke (CS)-induced airway inflammation was alleviated with LAT8881 treatment in an experimental COPD model over an 8 week period. Total number of cells in the BALF. Data are expressed as mean+/−SEM, and analysed using One-Way ANOVA, n=8, ****P<0.0001 and Mann-Whitney test, n=7-8, ###P<0.001.

FIG. 4A shows the increase in airway resistance (Rn) in mice challenged with an increasing concentration of methacholine. FIG. 4B shows that treatment with LAT8881 significantly reduced airway resistance when compared to sham (saline)-treated animals; n=8 per group, ***P<0.001; ****P<0.0001.

### DETAILED DESCRIPTION OF THE INVENTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

As used herein, the term “about” refers to a quantity, level, value, dimension, size, or amount that varies by as much as 10% (e.g., by 10%, 9%, 8%, 7%, 6%, 5%, 4%, 3%, 2% or 1%) to a reference quantity, level, value, dimension, size, or amount.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

### Peptides of Formula (I)

The present inventors has surprisingly found that peptides of formula (I) (SEQ ID NO:1) can alleviate at least some of the inflammatory mediators of inflammatory airway disease and reduce airway hyperresponsiveness. Thus, in an aspect disclosed herein, there is provided a method of treating an inflammatory airway disease in a subject, the method comprising administering to a subject in need thereof a therapeutically effective amount of a peptide of formula (I), or a pharmaceutically acceptable salt thereof:

(I)
(SEQ ID NO: 1)
R¹-CRSVEGSCG-R²

wherein
R¹ is selected from the group consisting of YLRIVQ (SEQ ID NO:45), LRIVQ (SEQ ID NO:46), RIVQ (SEQ ID NO:47), IVQ, VQ, and Q, or R¹ is absent; and R² is F (phenylalanine), or R² is absent.

In a preferred embodiment, the peptide is YLRIVQCRSVEGSCGF (SEQ ID NO:2). SEQ ID NO:2 (also referred to as AOD9604) is the C-terminal fragment of human growth hormone (hGH) spanning amino acid residues 178-192 of hGH (see, e.g., GenBank Accession numbers AAA72260.1, AML27053.1 and ADE06645.1), with an additional tyrosine residue at the N-terminus of the peptide.

In an embodiment disclosed herein, R¹ is absent. In another embodiment, R² is absent. In yet another embodiment, R¹ and R² are absent.

In an embodiment disclosed herein, the peptide of formula (I) is from 9 to 16 amino acid residues in length, preferably 9, 10, 11, 12, 13, 14, 15 or 16 amino acid residues in length. The peptide of formula (I) may comprise a disulphide bond between the two cysteine (C) residues, thereby forming a cyclic peptide between the two cysteine residues.

In an embodiment, the peptide of formula (I) is selected from the group consisting of YLRIVQCRSVEGSCGF (SEQ ID NO:2), LRIVQCRSVEGSCGF (SEQ ID NO:3), CRSVEGSCG (SEQ ID NO:4) and CRSVEGSCGF (SEQ ID NO:5).

In a preferred embodiment, the peptide of formula (I) is CRSVEGSCG (SEQ ID NO:4). In another preferred embodiment, the peptide of formula (I) is CRSVEGSCGF (SEQ ID NO:5).

### Peptides of Formula (II)

The present disclosure also extends to non-human variants of the peptides of formula (I) that have therapeutic properties for treating an inflammatory airway disease as their human counterparts. Suitable non-human variants of the peptides of formula (I) will be familiar to persons skilled in the art, illustrative examples of which are disclosed in WO 2013/082667, the contents of which is incorporated herein by reference. Thus, in an aspect disclosed herein, there is provided a method of treating an inflammatory airway disease in a subject, the method comprising administering to a subject in need thereof a therapeutically effective amount of a peptide of formula (II), or a pharmaceutically acceptable salt thereof:

(II)
(SEQ ID NO: 6)
R¹-CRRFVESSC-R²

wherein
R¹ is selected from the group consisting of YLRVMK (SEQ ID NO:48), LRVMK (SEQ ID NO:49), RVMK (SEQ ID NO:50), VMK, MK, and K, or R¹ is absent; and
R² is selected from the group consisting of A (alanine) and AF (alanine-phenylalanine), or R² is absent. The peptide of formula (II) is representative of a non-human variant of formula (I), as is found, for example in canine, equine and feline subjects.

In an embodiment, the peptide of formula (II) is selected from the group consisting of YLRVMKCRRFVESSCAF (SEQ ID NO:7), LRVMKCRRFVESSCAF (SEQ ID NO:8), CRRFVESSCAF (SEQ ID NO:9) and CRRFVESSCA (SEQ ID NO:10).

The peptide of formula (II) is from 9 to 17 amino acid residues in length, preferably 9, 10, 11, 12, 13, 14, 15, 16 or 17 amino acid residues in length. The peptide of formula (II) may comprise a disulphide bond between the two cysteine (C) residues, thereby forming a cyclic peptide between the two cysteine residues. In an embodiment, the peptide of formula (II) is selected from the group consisting of YLRVMKCRRFVESSCAF (SEQ ID NO:7), LRVMKCRRFVESSCAF (SEQ ID NO:8), CRRFVESSCAF (SEQ ID NO:9) and CRRFVESSCA (SEQ ID NO:10). In an embodiment, the peptide is YLRVMKCRRFVESSCAF (SEQ ID NO:7). In another embodiment, the peptide is CRRFVESSCAF (SEQ ID NO:9). In another embodiment, the peptide is CRRFVESSCA (SEQ ID NO:10).

### Peptides of Formula (III)

The present disclosure also extends to peptides of formula (III) as having therapeutic properties for the treatment of an inflammatory airway disease. Thus, in another aspect disclosed herein, there is provided a method of treating an inflammatory airway disease in a subject, the method comprising administering to a subject in need thereof a therapeutically effective amount of a peptide of formula (III):

(III)
(SEQ ID NO: 11)
R¹-C-R-X¹-X²-P-X³-X⁴-X⁵-X⁶-C-R²

wherein
X¹, X³, X⁵, and X⁶ is an amino acid residue selected from the group consisting of serine, alanine, valine, leucine, isoleucine and glycine;
X² is arginine or lysine;
X⁴ is glutamic acid or aspartic acid;
R¹ is selected from the group consisting of:
S,

(SEQ ID NO: 12)
HS,
(SEQ ID NO: 13)
GHS,
(SEQ ID NO: 14)
PGHS,
(SEQ ID NO: 15)
APGHS,
(SEQ ID NO: 16)
EAPGHS,
(SEQ ID NO: 17)
SEAPGHS,
(SEQ ID NO: 18)
SSEAPGHS,
(SEQ ID NO: 19)
PSSEAPGHS,
(SEQ ID NO: 20)
DPSSEAPGHS
and
(SEQ ID NO: 21)
IDPSSEAPGHS,

or R¹ is absent; and
R² is selected from the group consisting of
S,

(SEQ ID NO: 22)
SS,
(SEQ ID NO: 23)
SSK,
(SEQ ID NO: 24)
SSKF,
(SEQ ID NO: 25)
SSKFS,
(SEQ ID NO: 26)
SSKFSW,
(SEQ ID NO: 27)
SSKFSWD,
(SEQ ID NO: 28)
SSKFSWDE,
(SEQ ID NO: 29)
SSKFSWDEY,
(SEQ ID NO: 30)
SSKFSWDEYE,
(SEQ ID NO: 31)
SSKFSWDEYEQ,
(SEQ ID NO: 32)
SSKFSWDEYEQY,
(SEQ ID NO: 33)
SSKFSWDEYEQYK,
(SEQ ID NO: 34)
SSKFSWDEYEQYKK,
and
(SEQ ID NO: 35)
SSKFSWDEYEQYKKE,

or R² is absent.

In an embodiment, one or both of R¹ and R² further comprises polyethylene glycol (PEG). The PEG may have a molecular weight in the range of 220 to 5500 Da, preferably 220 to 2500 Da, or more preferably 570 to 1100 Da.

In an embodiment, R¹ is absent. In another embodiment, R² is absent. In yet another embodiment, R¹ and R² are absent.

In an embodiment, R¹ is capped with an N-terminal capping group. The term “N-terminal capping group” typically refers to a group that blocks the reactivity of the N-terminal amino group. Suitable N-terminal capping groups will be familiar to persons skilled in the art, illustrative examples of which include acyl groups that form amide groups with the N-terminal amino group, for example, the N-terminal capping group forms a —NHC(O)Ra, where the NH is from the N-terminal amino group and Ra is alkyl, alkenyl, alkynyl, cycloalkyl or aryl. In an embodiment, the N-terminal capping group is —C(O)CH₃ (acyl), forming —NHC(O)CH₃.

In an embodiment, R¹ is a serine residue (S).

In another embodiment, R² is capped with an C-terminal capping group. The term “C-terminal capping group” typically refers to a group that blocks the reactivity of the C-terminal carboxylic acid. Suitable C-terminal capping groups form amide groups or esters with the C-terminal carboxylic acid, for example, the C-terminal capping group forms a —C(O)NHR^{a} or —C(O)OR^{b}, where the C(O) is from the C-terminal carboxylic acid group and R^{a} is hydrogen, alkyl, alkenyl, alkynyl, cycloalkyl or aryl and R^{b} is alkyl, alkenyl, alkynyl, cycloalkyl or aryl. In particular embodiments, the C-terminal capping group is —NH₂, forming —C(O)NH₂.

In an embodiment, R² is a serine residue (S).

In another embodiment, R¹ is a serine residue and R² is a serine residue.

The peptides of formula (III) can be from 10 to 50 amino acid residues in length (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acid residues in length), preferably 10 to 40 in length, more preferably 10 to 30 in length, more preferably 10 to 25 in length, or more preferably 10 to 20 in length. It is to be understood that a cyclic peptide, as herein described, is one in which the side chains of two amino acid residues (typically cysteine residues) react together to form a covalent bond or in which the C-terminal carboxylic acid and the N-terminal amine group form an amide bond, thereby cyclizing the peptide.

In an embodiment disclosed herein, the peptide of formula (III) has an amino acid sequence selected from the group consisting of:

(SEQ ID NO: 36)
SCRSRPVESSC;
(SEQ ID NO: 37)
CRSRPVESSC;
(SEQ ID NO: 38)
CRSRPVESSCS;
and
(SEQ ID NO: 39)
SCRSRPVESSCS.

### Peptides of Formula (IV)

The present disclosure also extends to peptides of formula (IV) as having therapeutic properties for the treatment of an inflammatory airway disease. Thus, in an aspect disclosed herein, there is provided a method of treating an inflammatory airway disease in a subject, the method comprising administering to a subject in need thereof a therapeutically effective amount of a peptide of formula (IV) or a pharmaceutically acceptable salt thereof:

(IV)
(SEQ ID NO: 40)
R¹-C-R-I-X¹-X²-X³-X⁴-N-C-R²

wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK (SEQ ID NO:51), LKLLK (SEQ ID NO:52), KLLK (SEQ ID NO:53, LLK, LK, K or R¹ is absent; and
R² is G (glycine), or R² is absent, or R² is a pharmaceutically acceptable carrier.

In an embodiment, the peptide of formula (IV) is selected from the group consisting of amino acid sequence CRIIHNNNC (SEQ ID NO:41), CRIIHNNNCG (SEQ ID NO:42), CRIVYDSNC (SEQ ID NO:43) and CRIVYDSNCG (SEQ ID NO:44). In a preferred embodiment, the peptide of formula (IV) is CRIIHNNNC (SEQ ID NO:41). SEQ ID NO:41 (also referred to interchangeably herein as LAT7771) is the C-terminal fragment of human prolactin (PRL) spanning amino acid residues 219-227 of human prolactin precursor (hPRL; see, e.g., NCBI Reference sequence NP_000939.1 and NP_001157030).

The peptides of formulae (I), (II), (III) and (IV) may be made of naturally occurring amino acid residues, proteogenic or non-proteogenic. These amino acids have L-stereochemistry. Naturally occurring amino acids are set out in Table 1, below.

TABLE 1
(1)

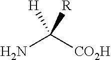

(2)

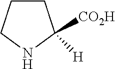

| | Three-letter | One-letter | Structure of side chain |
|---|---|---|---|
| Amino Acid | Abbreviation | symbol | (R) in (1) above |
| Alanine | Ala | A | —CH₃ |
| Arginine | Arg | R | —(CH₂)₃NHC(═N)NH₂ |
| Asparagine | Asn | N | —CH₂CONH₂ |
| Aspartic acid | Asp | D | —CH₂CO₂H |
| Cysteine | Cys | C | —CH₂SH |
| Glutamine | Gln | Q | —(CH₂)₂CONH₂ |
| Glutamic acid | Glu | E | —(CH₂)₂CO₂H |
| Glycine | Gly | G | —H |
| Histidine | His | H | —CH₂(4-imidazoly1) |
| Isoleucine | Ile | I | —CH(CH₃)CH₂CH₃ |
| Leucine | Leu | L | —CH₂CH(CH₃)₂ |
| Lysine | Lys | K | —(CH₂)₄NH₂ |
| Methionine | Met | M | —(CH₂)₂SCH₃ |
| Phenylalanine | Phe | F | —CH₂Ph |
| Ornithine | Orn | O | —(CH₂)₃NH₂ |
| Proline | Pro | P | see formula (2) above for |
| | | | structure of amino acid |
| Serine | Ser | S | —CH₂OH |
| Threonine | Thr | T | —CH(CH₃)OH |
| Tryptophan | Trp | W | —CH₂(3-indoly1) |
| Tyrosine | Tyr | Y | —CH₂(4-hydroxyphenyl) |
| Valine | Val | V | —CH(CH₃)₂ |

As used herein, the term “alkyl” refers to a straight chain or branched saturated hydrocarbon group having 1 to 10 carbon atoms. Where appropriate, the alkyl group may have a specified number of carbon atoms, for example, C₁₋₆alkyl which includes alkyl groups having 1, 2, 3, 4, 5 or 6 carbon atoms in a linear or branched arrangement. Examples of suitable alkyl groups include, but are not limited to, methyl, ethyl, n-propyl, i-propyl, n-butyl, i-butyl, t-butyl, n-pentyl, 2-methylbutyl, 3-methylbutyl, 4-methylbutyl, n-hexyl, 2-methylpentyl, 3-methylpentyl, 4-methylpentyl, 5-methylpentyl, 2-ethylbutyl, 3-ethylbutyl, heptyl, octyl, nonyl and decyl.

As used herein, the term “alkenyl” refers to a straight-chain or branched hydrocarbon group having one or more double bonds between carbon atoms and having 2 to 10 carbon atoms. Where appropriate, the alkenyl group may have a specified number of carbon atoms. For example, C₂-C₆ as in “C₂-C₆alkenyl” includes groups having 2, 3, 4, 5 or 6 carbon atoms in a linear or branched arrangement. Examples of suitable alkenyl groups include, but are not limited to, ethenyl, propenyl, isopropenyl, butenyl, butadienyl, pentenyl, pentadienyl, hexenyl, hexadienyl, heptenyl, octenyl, nonenyl and decenyl.

As used herein, the term “alkynyl” refers to a straight-chain or branched hydrocarbon group having one or more triple bonds and having 2 to 10 carbon atoms. Where appropriate, the alkynyl group may have a specified number of carbon atoms. For example, C₂-C₆ as in “C₂-C₆alkynyl” includes groups having 2, 3, 4, 5 or 6 carbon atoms in a linear or branched arrangement. Examples of suitable alkynyl groups include, but are not limited to ethynyl, propynyl, butynyl, pentynyl and hexynyl.

As used herein, the term “cycloalkyl” refers to a saturated and unsaturated (but not aromatic) cyclic hydrocarbon. The cycloalkyl ring may include a specified number of carbon atoms. For example, a 3 to 8 membered cycloalkyl group includes 3, 4, 5, 6, 7 or 8 carbon atoms. Examples of suitable cycloalkyl groups include, but are not limited to, cyclopropyl, cyclobutyl, cyclopentyl, cyclopentenyl, cyclohexyl, cyclohexenyl, cyclohexadienyl, cycloheptyl and cyclooctyl.

As used herein, the term “aryl” is intended to mean any stable, monocyclic, bicyclic or tricyclic carbon ring system of up to 7 atoms in each ring, wherein at least one ring is aromatic. Examples of such aryl groups include, but are not limited to, phenyl, naphthyl, tetrahydronaphthyl, indanyl, fluorenyl, phenanthrenyl, biphenyl and binaphthyl.

In an embodiment, a disulphide bond is formed between the two cysteine residues (C) of formulae (I), (II) and (III).

The peptides disclosed herein may be made by methods well known to persons skilled in the art, illustrative examples of which include by solution or solid phase synthesis using Fmoc or Boc protected amino acid residues and recombinant techniques as known in the art using standard microbial culture technology, genetically engineered microbes and recombinant DNA technology (Sambrook and Russell, Molecular Cloning: A Laboratory Manual (3^{rd} Edition), 2001, CSHL Press).

In an embodiment, the peptides of formulae (I), (II), (III) and (IV) are formed as a pharmaceutically acceptable salt. It is to be understood that non-pharmaceutically acceptable salts are also envisaged, since these may be useful as intermediates in the preparation of pharmaceutically acceptable salts or may be useful during storage or transport. Suitable pharmaceutically acceptable salts will be familiar to persons skilled in the art, illustrative examples of which include salts of pharmaceutically acceptable inorganic acids, such as hydrochloric, sulphuric, phosphoric, nitric, carbonic, boric, sulfamic, and hydrobromic acids, or salts of pharmaceutically acceptable organic acids, such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicylic sulphanilic, aspartic, glutamic, edetic, stearic, palmitic, oleic, lauric, pantothenic, tannic, ascorbic and valeric acids. Illustrative examples of suitable base salts include those formed with pharmaceutically acceptable cations, such as sodium, potassium, lithium, calcium, magnesium, ammonium and alkylammonium. Basic nitrogen-containing groups may be quaternized with such agents as lower alkyl halide, such as methyl, ethyl, propyl, and butyl chlorides, bromides and iodides; dialkyl sulfates like dimethyl and diethyl sulfate; and others.

Also disclosed herein are prodrugs comprising the peptides of formulae (I), (II), (III) or (IV), or the pharmaceutically acceptable salts thereof. As used herein, a “prodrug” typically refers to a compound that can be metabolized in vivo to provide the active peptide of formulae (I), (II), (III) or (IV), or pharmaceutically acceptable salts thereof. In some embodiments, the prodrug itself also shares the same, or substantially the same, therapeutic activity as the peptide of formulae (I), (II), (III) or (IV), or pharmaceutically acceptable salts thereof, as described elsewhere herein.

In some embodiments, the peptides of formulae (I), (II), (III) and (IV), or pharmaceutically acceptable salts thereof, may further comprise a C-terminal capping group. The term “C-terminal capping group”, as used herein, refers to a group that blocks the reactivity of the C-terminal carboxylic acid. Suitable C-terminal capping groups form amide groups or esters with the C-terminal carboxylic acid, for example, the C-terminal capping group forms a —C(O)NHR^{a} or —C(O)OR^{b} where the C(O) is from the C-terminal carboxylic acid group and R^{a} is hydrogen, alkyl, alkenyl, alkynyl, cycloalkyl or aryl and R^{b} is alkyl, alkenyl, alkynyl, cycloalkyl or aryl. In particular embodiments, the C-terminal capping group is —NH₂, forming —C(O)NH₂. In some embodiments, the peptides of formulae (I) or (II), or pharmaceutically acceptable salts thereof, comprise a C-terminal polyethylene glycol (PEG). In an embodiment, the PEG has a molecular weight in the range of 220 to 5500 Da, preferably 220 to 2500 Da, more preferably 570 to 1100 Da.

In some embodiments, the peptides of formulae (I), (II), (III) and (IV), or pharmaceutically acceptable salts thereof, may further comprise an N-terminal capping group. The term “N-terminal capping group”, as used herein, refers to a group that blocks the reactivity of the N-terminal amino group. Suitable N-terminal capping groups are acyl groups that form amide groups with the N-terminal amino group, for example, the N-terminal capping group forms a —NHC(O)R^{a} where the NH is from the N-terminal amino group and R^{a} is alkyl, alkenyl, alkynyl, cycloalkyl or aryl. In particular embodiments, the N-terminal capping group is —C(O)CH₃ (acyl), forming —NHC(O)CH₃.

In some embodiments, the peptides of formulae (I), (II), (III) and (IV), or pharmaceutically acceptable salts thereof, may comprise a C-terminal capping group and an N-terminal capping group, as herein described. It is to be understood that the peptides disclosed herein do not include the full length amino acid sequence of human growth hormone or of a non-human isoform thereof.

The peptides of formulae (I), (II), (III) and (IV), or pharmaceutically acceptable salts thereof, as herein described, can be made be any method known to persons skilled in the art. Illustrative examples of suitable methods include solution or solid phase synthesis using Fmoc or Boc protected amino acid residues, recombinant techniques using microbial culture, genetically engineered microbes, plants and recombinant DNA technology (see, e.g., Sambrook and Russell, Molecular Cloning: A Laboratory Manual (3^{rd} Edition), 2001, CSHL Press).

### Methods of Treatment

As described elsewhere herein, the present inventor has surprisingly found, for the first time, that a peptide of formula (I) (SEQ ID NO:1) can alleviate inflammation in inflammatory airway disease and reduce airway hyperresponsiveness. The peptides of formula (I) can therefore suitably be used to treat, alleviate or otherwise abrogate the severity of an inflammatory airway disease in a subject, including one or more symptoms thereof. The present disclosure also extends to the use of formulae (II), (III) and (IV) for treating an inflammatory airway disease. Thus, the peptides of formulae (I), (II), (III) and (IV), or pharmaceutically acceptable salts thereof, can also suitably be used to treat, alleviate or otherwise abrogate the severity of an inflammatory airway disease in a subject.

The terms “treating”, “treatment” and the like, are used interchangeably herein to mean relieving, reducing, alleviating, ameliorating or otherwise inhibiting the severity of the inflammatory airway disease, including one or more symptoms thereof. The terms “treating”, “treatment” and the like are also used interchangeably herein to mean preventing the inflammatory airway disease, including one or more symptoms thereof.

The terms “treating”, “treatment” and the like also include preventing, relieving, reducing, alleviating, ameliorating or otherwise inhibiting the severity of an inflammatory airway disease for at least a period of time. It is to be understood that the terms “treating”, “treatment” and the like do not imply that the inflammatory airway disease, or a symptom thereof, is permanently prevented, relieved, reduced, alleviated, ameliorated or otherwise inhibited and therefore extend to the temporary prevention, relief, reduction, alleviation, amelioration or otherwise inhibition of the severity of the inflammatory airway disease, or of one or more symptoms thereof.

The term “subject”, as used herein, refers to a mammalian subject for whom treatment of an inflammatory airway disease is desired. Illustrative examples of suitable subjects include primates, especially humans, companion animals such as cats and dogs and the like, working animals such as horses, donkeys and the like, livestock animals such as sheep, cows, goats, pigs and the like, laboratory test animals such as rabbits, mice, rats, guinea pigs, hamsters and the like and captive wild animals such as those in zoos and wildlife parks, deer, dingoes and the like. In an embodiment, the subject is a human.

It is to be understood that a reference to a subject herein does not imply that the subject has an inflammatory airway disease, or a symptom thereof, but also includes a subject that is at risk of developing an inflammatory airway disease, or a symptom thereof.

In an embodiment, the methods disclosed herein comprise administering a peptide of formula (I), (II), (III) or (IV), or a pharmaceutically acceptable salt thereof, to a human subject.

The peptides of formula (I), (II), (III) and (IV), or pharmaceutically acceptable salts thereof, are to be administered in a therapeutically effective amount. The phrase “therapeutically effective amount” typically means an amount necessary to attain the desired response. It would be understood by persons skilled in the art that the therapeutically effective amount of peptide will vary depending upon several factors, illustrative examples of which include the health and physical condition of the subject to be treated, the taxonomic group of subject to be treated, the severity of the inflammatory airway disease to be treated, the formulation of the composition comprising a peptide of formula (I), (II), (III) or (IV), or a pharmaceutically acceptable salt thereof, the route of administration, and combinations of any of the foregoing.

The therapeutically effective amount will typically fall within a relatively broad range that can be determined through routine trials by persons skilled in the art. Illustrative examples of a suitable therapeutically effective amount of the peptides of formula (I), (II), (III) and (IV), and pharmaceutically acceptable salts thereof, for administration to a human subject include from about 0.001 mg per kg of body weight to about 1 g per kg of body weight, preferably from about 0.001 mg per kg of body weight to about 50 g per kg of body weight, more preferably from about 0.01 mg per kg of body weight to about 1.0 mg per kg of body weight. In an embodiment disclosed herein, the therapeutically effective amount of the peptide of formulae (I), (II), (III) and/or (IV), and/or pharmaceutically acceptable salts thereof, is from about 0.001 mg per kg of body weight to about 1 g per kg of body weight per dose (e.g., 0.001 mg/kg, 0.005 mg/kg, 0.01 mg/kg, 0.05 mg/kg, 0.1 mg/kg, 0.15 mg/kg, 0.2 mg/kg, 0.25 mg/kg, 0.3 mg/kg, 0.35 mg/kg, 0.4 mg/kg, 0.45 mg/kg, 0.5 mg/kg, 0.5 mg/kg, 0.55 mg/kg, 0.6 mg/kg, 0.65 mg/kg, 0.7 mg/kg, 0.75 mg/kg, 0.8 mg/kg, 0.85 mg/kg, 0.9 mg/kg, 0.95 mg/kg, 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 15.5 mg/kg, 16 mg/kg, 16.5 mg/kg, 17 mg/kg, 17.5 mg/kg, 18 mg/kg, 18.5 mg/kg, 19 mg/kg, 19.5 mg/kg, 20 mg/kg, 20.5 mg/kg, 21 mg/kg, 21.5 mg/kg, 22 mg/kg, 22.5 mg/kg, 23 mg/kg, 23.5 mg/kg, 24 mg/kg, 24.5 mg/kg, 25 mg/kg, 25.5 mg/kg, 26 mg/kg, 26.5 mg/kg, 27 mg/kg, 27.5 mg/kg, 28 mg/kg, 28.5 mg/kg, 29 mg/kg, 29.5 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg of body weight, etc). In an embodiment, the therapeutically effective amount of the peptides of formulae (I), (II), (III) or (IV), or the pharmaceutically acceptable salts thereof, is from about 0.001 mg to about 50 mg per kg of body weight. In an embodiment, the therapeutically effective amount of the peptide of formula (I), (II), (III) or (IV), and pharmaceutically acceptable salts thereof, is from about 0.01 mg to about 100 mg per kg of body weight. In an embodiment, the therapeutically effective amount of the peptide of formula (I), (II), (III) or (IV), or pharmaceutically acceptable salts thereof, is from about 0.1 mg to about 10 mg per kg of body weight, preferably from about 0.1 mg to about 5 mg per kg of body weight, more preferably from about 0.1 mg to about 1.0 mg per kg of body weight. Dosage regimes may be adjusted to provide the optimum therapeutic response. For example, several divided doses may be administered daily, weekly, monthly or other suitable time intervals, or the dose may be proportionally reduced as indicated by the exigencies of the situation.

As noted elsewhere herein, the present inventor has surprisingly found that the peptides described herein are capable of reducing the inflammatory mediators or markers of inflammatory airway disease and reduce airway hyperresponsiveness. Thus, in an embodiment disclosed herein, a peptide of formula (I), or a pharmaceutically acceptable salt thereof, is administered to the subject at a therapeutically effective amount that treats an inflammatory airway disease in the subject. Therapeutic activity in treating an inflammatory airway disease is also ascribed to the peptides of formulae (II), (III) and (IV). Thus, in an embodiment disclosed herein, a peptide of formula (II), (III) or (IV), or pharmaceutically acceptable salts thereof, is administered to the subject at a therapeutically effective amount that treats an inflammatory airway disease in the subject.

In an embodiment disclosed herein, the peptides described herein comprise the amino acid sequence CRSVEGSCG (SEQ ID NO:4) or CRSVEGSCGF (SEQ ID NO:5).

### Inflammatory Airway Disease

Inflammatory airway diseases will be familiar to persons skilled in the art, illustrative examples of which include chronic obstructive pulmonary disease (COPD), asthma, chronic bronchitis, emphysema, cystic fibrosis, lung cancer and bronchopulmonary dysplasia. In an embodiment, the inflammatory airway disease is COPD. In an embodiment, the inflammatory airway disease is asthma. In an embodiment, the inflammatory airway disease is chronic bronchitis. In an embodiment, the inflammatory airway disease is emphysema. In an embodiment, the inflammatory airway disease is cystic fibrosis. In an embodiment, the inflammatory airway disease is associated with lung cancer. In an embodiment, the inflammatory airway disease is bronchopulmonary dysplasia.

The methods, compositions and uses thereof, as described herein, may be particularly useful for treating an inflammatory airway disease in a subject that is susceptible to a condition that would otherwise exacerbate the inflammatory airway disease. Such underlying conditions will be known to persons skilled in the art, illustrative examples of which include respiratory infection by, e.g., viruses, bacteria or other pathogens. In another embodiment, the subject is immunocompromised, whether as a result of treatment (e.g., by chemotherapy, radiotherapy) or otherwise (e.g., by HIV infection).

### Routes of Administration

The peptides of formulae (I), (II), (III) and (IV), and pharmaceutically acceptable salts thereof, may be administered to the subject by any suitable route that allows for delivery of the peptides to the subject at a therapeutically effective amount, as herein described. Suitable routes of administration will be known to persons skilled in the art, illustrative examples of which include enteral routes of administration (e.g., oral and rectal), parenteral routes of administration, typically by injection or microinjection (e.g., intramuscular, subcutaneous, intravenous, epidural, intra-articular, intraperitoneal, intracisternal or intrathecal) and topical (transdermal or transmucosal) routes of administration (e.g., buccal, sublingual, vaginal, intranasal or by inhalation, insufflation, suppository or nebulization). In an embodiment, the route of administration is by inhalation or insufflation. The peptides of formulae (I), (II), (III) and (IV), and pharmaceutically acceptable salts thereof, may also suitably be administered to the subject as a controlled release dosage form to provide a controlled release of the active agent(s) over an extended period of time. The term “controlled release” typically means the release of the active agent(s) to provide a constant, or substantially constant, concentration of the active agent in the subject over a period of time (e.g., about eight hours up to about 12 hours, up to about 14 hours, up to about 16 hours, up to about 18 hours, up to about 20 hours, up to a day, up to a week, up to a month, or more than a month). Controlled release of the active agent(s) can begin within a few minutes after administration or after expiration of a delay period (lag time) after administration, as may be required. Suitable controlled release dosage forms will be known to persons skilled in the art, illustrative examples of which are described in Anal, A. K. (2010; Controlled-Release Dosage Forms. Pharmaceutical Sciences Encyclopedia. 11:1-46).

Without being bound by theory or by a particular mode of application, it may be desirable to elect a route of administration on the basis of the severity of the inflammatory airway disease or one or more symptoms thereof. In an embodiment disclosed herein, the peptides of formulae (I), (II), (III) and (IV), or pharmaceutically acceptable salts thereof, are administered to the subject enterally. In an embodiment disclosed herein, the peptides of formula (I), (II), (III) or (IV), or pharmaceutically acceptable salts thereof, are administered to the subject orally. In an embodiment disclosed herein, the peptides of formula (I), (II), (III) or (IV), or pharmaceutically acceptable salts thereof, are administered to the subject parenterally. In another embodiment disclosed herein, the peptides of formula (I), (II), (III) or (IV), or pharmaceutically acceptable salts thereof, are administered to the subject topically. In another embodiment disclosed herein, the peptides of formula (I), (II), (III) or (IV), or pharmaceutically acceptable salts thereof, are administered to the subject by inhalation. In another embodiment disclosed herein, the peptides of formula (I), (II), (III) or (IV), or pharmaceutically acceptable salts thereof, are administered to the subject by insufflation.

As described elsewhere herein, “topical” administration typically means application of the active agents to a surface of the body, such as the skin or mucous membranes, suitably in the form of a cream, lotion, foam, gel, ointment, nasal drop, eye drop, ear drop, transdermal patch, transdermal film (e.g., sublingual film) and the like. Topical administration also encompasses administration via the mucosal membrane of the respiratory tract by inhalation or insufflation. In an embodiment disclosed herein, the topical administration is selected from the group consisting of transdermal and transmucosal administration. In an embodiment, the peptides of formula (I), (II), (III) or (IV), or pharmaceutically acceptable salts thereof, are administered to the subject transdermally. In an embodiment, the peptides of formulae (I), (II), (III) and (IV), and pharmaceutically acceptable salts thereof, are administered to the subject by inhalation, insufflation or nebulization.

In an embodiment, the methods comprise administering the peptide of formula (I), or a pharmaceutically acceptable salt thereof, to a human by inhalation or insufflation. In another embodiment, the methods comprise administering the peptide of formula (I), or pharmaceutically acceptable salts thereof, to a non-human subject by inhalation or insufflation. In yet another embodiment, the methods comprise administering the peptide of formula (I), or a pharmaceutically acceptable salt thereof, by inhalation or insufflation, to a non-human subject selected from the group consisting of a feline, a canine and an equine.

In an embodiment, the methods comprise administering the peptide of formula (II), or a pharmaceutically acceptable salt thereof, to a human by inhalation or insufflation. In another embodiment, the methods comprise administering the peptide of formula (II), or a pharmaceutically acceptable salt thereof, to a non-human subject by inhalation or insufflation. In yet another embodiment, the methods comprise administering the peptide of formula (II), or a pharmaceutically acceptable salt thereof, by inhalation or insufflation, to a non-human subject selected from the group consisting of a feline, a canine and an equine.

In an embodiment, the methods comprise administering the peptide of formula (III), or a pharmaceutically acceptable salt thereof, to a human by inhalation or insufflation. In another embodiment, the methods comprise administering the peptide of formula (III), or a pharmaceutically acceptable salt thereof, to a non-human subject by inhalation or insufflation. In yet another embodiment, the methods comprise administering the peptide of formula (III), or a pharmaceutically acceptable salt thereof, by inhalation or insufflation, to a non-human subject selected from the group consisting of a feline, a canine and an equine.

In an embodiment, the methods comprise administering the peptide of formula (IV), or a pharmaceutically acceptable salt thereof, to a human by inhalation or insufflation. In another embodiment, the methods comprise administering the peptide of formula (IV), or a pharmaceutically acceptable salt thereof, to a non-human subject by inhalation or insufflation. In yet another embodiment, the methods comprise administering the peptide of formula (IV), or a pharmaceutically acceptable salt thereof, by inhalation or insufflation, to a non-human subject selected from the group consisting of a feline, a canine and an equine.

In an embodiment, the methods comprise administering the peptide of formula (I), or a pharmaceutically acceptable salt thereof, orally to a human. In another embodiment, the methods comprise administering the peptide of formula (I), or a pharmaceutically acceptable salt thereof, orally to a non-human subject. In yet another embodiment, the methods comprise administering the peptide of formula (I), or a pharmaceutically acceptable salt thereof, orally to a non-human subject selected from the group consisting of a feline, a canine and an equine.

In an embodiment, the methods comprise administering the peptide of formula (II), or a pharmaceutically acceptable salt thereof, orally to a human. In another embodiment, the methods comprise administering the peptide of formula (II), or a pharmaceutically acceptable salt thereof, orally to a non-human subject. In yet another embodiment, the methods comprise administering the peptide of formula (II), or a pharmaceutically acceptable salt thereof, orally to a non-human subject selected from the group consisting of a feline, a canine and an equine.

In an embodiment, the methods comprise administering the peptide of formula (III), or a pharmaceutically acceptable salt thereof, orally to a human. In another embodiment, the methods comprise administering the peptide of formula (III), or a pharmaceutically acceptable salt thereof, orally to a non-human subject. In yet another embodiment, the methods comprise administering the peptide of formula (III), or a pharmaceutically acceptable salt thereof, orally to a non-human subject selected from the group consisting of a feline, a canine and an equine.

In an embodiment, the methods comprise administering the peptide of formula (IV), or a pharmaceutically acceptable salt thereof, orally to a human. In another embodiment, the methods comprise administering the peptide of formula (IV), or a pharmaceutically acceptable salt thereof, orally to a non-human subject. In yet another embodiment, the methods comprise administering the peptide of formula (IV), or a pharmaceutically acceptable salt thereof, orally to a non-human subject selected from the group consisting of a feline, a canine and an equine.

In another embodiment, the methods comprise administering the peptide of SEQ ID NO:2, or a pharmaceutically acceptable salt thereof, orally to a human. In another embodiment, the methods comprise administering the peptide of SEQ ID NO:2, or pharmaceutically acceptable salts thereof, orally to a non-human subject. In yet another embodiment, the methods comprise administering the peptide of SEQ ID NO:2, or pharmaceutically acceptable salts thereof, orally to a non-human subject selected from the group consisting of a feline, a canine and an equine.

In another embodiment, the methods comprise administering the peptide of SEQ ID NO:2, or a pharmaceutically acceptable salt thereof, topically to a human. In another embodiment, the methods comprise administering the peptide of SEQ ID NO:2, or pharmaceutically acceptable salts thereof, topically to a non-human subject. In yet another embodiment, the methods comprise administering the peptide of SEQ ID NO:2, or pharmaceutically acceptable salts thereof, topically to a non-human subject selected from the group consisting of a feline, a canine and an equine.

In another embodiment, the methods comprise administering the peptide of SEQ ID NO:2, or a pharmaceutically acceptable salt thereof, to a human by inhalation of insufflation. In another embodiment, the methods comprise administering the peptide of SEQ ID NO:2, or pharmaceutically acceptable salts thereof, to a non-human subject by inhalation of insufflation. In yet another embodiment, the methods comprise administering the peptide of SEQ ID NO:2, or pharmaceutically acceptable salts thereof, by inhalation of insufflation, to a non-human subject selected from the group consisting of a feline, a canine and an equine.

In another embodiment, the methods comprise administering the peptide of SEQ ID NO:7, or pharmaceutically acceptable salts thereof, orally to a non-human subject. In yet another embodiment, the methods comprise administering the peptide of SEQ ID NO:7, or pharmaceutically acceptable salts thereof, orally to a non-human subject selected from the group consisting of a feline, a canine and an equine.

In another embodiment, the methods comprise administering the peptide of SEQ ID NO:7, or pharmaceutically acceptable salts thereof, topically to a non-human subject. In yet another embodiment, the methods comprise administering the peptide of SEQ ID NO:7, or pharmaceutically acceptable salts thereof, topically to a non-human subject selected from the group consisting of a feline, a canine and an equine.

In another embodiment, the methods comprise administering the peptide of SEQ ID NO:7, or a pharmaceutically acceptable salt thereof, to a human by inhalation of insufflation. In another embodiment, the methods comprise administering the peptide of SEQ ID NO:7, or pharmaceutically acceptable salts thereof, to a non-human subject by inhalation of insufflation. In yet another embodiment, the methods comprise administering the peptide of SEQ ID NO:7, or pharmaceutically acceptable salts thereof, by inhalation of insufflation, to a non-human subject selected from the group consisting of a feline, a canine and an equine.

In another embodiment, the methods comprise administering the peptide of SEQ ID NO:36, or pharmaceutically acceptable salts thereof, orally to a non-human subject. In yet another embodiment, the methods comprise administering the peptide of SEQ ID NO:36, or pharmaceutically acceptable salts thereof, orally to anon-human subject selected from the group consisting of a feline, a canine and an equine.

In another embodiment, the methods comprise administering the peptide of SEQ ID NO:36, or pharmaceutically acceptable salts thereof, topically to a non-human subject. In yet another embodiment, the methods comprise administering the peptide of SEQ ID NO:36, or pharmaceutically acceptable salts thereof, topically to a non-human subject selected from the group consisting of a feline, a canine and an equine.

In another embodiment, the methods comprise administering the peptide of SEQ ID NO:36, or a pharmaceutically acceptable salt thereof, to a human by inhalation of insufflation. In another embodiment, the methods comprise administering the peptide of SEQ ID NO:36, or pharmaceutically acceptable salts thereof, to a non-human subject by inhalation of insufflation. In yet another embodiment, the methods comprise administering the peptide of SEQ ID NO:36, or pharmaceutically acceptable salts thereof, by inhalation of insufflation, to a non-human subject selected from the group consisting of a feline, a canine and an equine.

In another embodiment, the methods comprise administering the peptide of SEQ ID NO:41, or pharmaceutically acceptable salts thereof, orally to a non-human subject. In yet another embodiment, the methods comprise administering the peptide of SEQ ID NO:41, or pharmaceutically acceptable salts thereof, orally to a non-human subject selected from the group consisting of a feline, a canine and an equine.

In another embodiment, the methods comprise administering the peptide of SEQ ID NO:41, or pharmaceutically acceptable salts thereof, topically to a non-human subject. In yet another embodiment, the methods comprise administering the peptide of SEQ ID NO:41, or pharmaceutically acceptable salts thereof, topically to a non-human subject selected from the group consisting of a feline, a canine and an equine.

In another embodiment, the methods comprise administering the peptide of SEQ ID NO:41, or a pharmaceutically acceptable salt thereof, to a human by inhalation of insufflation. In another embodiment, the methods comprise administering the peptide of SEQ ID NO:41, or pharmaceutically acceptable salts thereof, to a non-human subject by inhalation of insufflation. In yet another embodiment, the methods comprise administering the peptide of SEQ ID NO:41, or pharmaceutically acceptable salts thereof, by inhalation of insufflation, to a non-human subject selected from the group consisting of a feline, a canine and an equine.

Illustrative examples of topical administration are described elsewhere herein. In an embodiment, the topical administration is transdermal.

In an embodiment disclosed herein, the peptides of formula (I), (II), (III) or (IV), or pharmaceutically acceptable salts thereof, are administered to the subject as a controlled release dosage form, illustrative examples of which are described elsewhere herein. In an embodiment, the methods comprise administering the peptide of formula (I), or a pharmaceutically acceptable salt thereof, to a human as a controlled release dosage form. In another embodiment, the methods comprise administering the peptide of formula (I), or pharmaceutically acceptable salts thereof, to a non-human subject as a controlled release dosage form. In yet another embodiment, the methods comprise administering the peptide of formula (I), or a pharmaceutically acceptable salt thereof, as a controlled release dosage form to a non-human subject selected from the group consisting of a feline, a canine and an equine.

In another embodiment, the methods comprise administering the peptide of formula (II), or a pharmaceutically acceptable salt thereof, to a human as a controlled release dosage form. In another embodiment, the methods comprise administering the peptide of formula (II), or a pharmaceutically acceptable salt thereof, to a non-human subject as a controlled release dosage form. In yet another embodiment, the methods comprise administering the peptide of formula (II), or a pharmaceutically acceptable salt thereof, as a controlled release dosage form to a non-human subject selected from the group consisting of a feline, a canine and an equine.

In another embodiment, the methods comprise administering the peptide of formula (III), or a pharmaceutically acceptable salt thereof, to a human as a controlled release dosage form. In another embodiment, the methods comprise administering the peptide of formula (III), or a pharmaceutically acceptable salt thereof, to a non-human subject as a controlled release dosage form. In yet another embodiment, the methods comprise administering the peptide of formula (III), or a pharmaceutically acceptable salt thereof, as a controlled release dosage form to a non-human subject selected from the group consisting of a feline, a canine and an equine.

In another embodiment, the methods comprise administering the peptide of formula (IV), or a pharmaceutically acceptable salt thereof, to a human as a controlled release dosage form. In another embodiment, the methods comprise administering the peptide of formula (IV), or a pharmaceutically acceptable salt thereof, to a non-human subject as a controlled release dosage form. In yet another embodiment, the methods comprise administering the peptide of formula (IV), or a pharmaceutically acceptable salt thereof, as a controlled release dosage form to a non-human subject selected from the group consisting of a feline, a canine and an equine.

In another embodiment, the methods comprise administering the peptide of SEQ ID NO:2, or a pharmaceutically acceptable salt thereof, to a human as a controlled release dosage form. In another embodiment, the methods comprise administering the peptide of SEQ ID NO:2, or pharmaceutically acceptable salts thereof, to a non-human subject as a controlled release dosage form. In yet another embodiment, the methods comprise administering the peptide of SEQ ID NO:2, or pharmaceutically acceptable salts thereof, as a controlled release dosage form to a non-human subject selected from the group consisting of a feline, a canine and an equine.

In another embodiment, the methods comprise administering the peptide of SEQ ID NO:7, or pharmaceutically acceptable salts thereof, to a non-human subject as a controlled release dosage form. In yet another embodiment, the methods comprise administering the peptide of SEQ ID NO:7, or pharmaceutically acceptable salts thereof, as a controlled release dosage form to a non-human subject selected from the group consisting of a feline, a canine and an equine. In an embodiment, the controlled release dosage form is administered to the subject parenterally, suitable examples of which are described elsewhere herein.

In another embodiment, the methods comprise administering the peptide of SEQ ID NO:36, or pharmaceutically acceptable salts thereof, to a non-human subject as a controlled release dosage form. In yet another embodiment, the methods comprise administering the peptide of SEQ ID NO:36, or pharmaceutically acceptable salts thereof, as a controlled release dosage form to a non-human subject selected from the group consisting of a feline, a canine and an equine. In an embodiment, the controlled release dosage form is administered to the subject parenterally, suitable examples of which are described elsewhere herein.

In another embodiment, the methods comprise administering the peptide of SEQ ID NO:41, or pharmaceutically acceptable salts thereof, to a non-human subject as a controlled release dosage form. In yet another embodiment, the methods comprise administering the peptide of SEQ ID NO:41, or pharmaceutically acceptable salts thereof, as a controlled release dosage form to a non-human subject selected from the group consisting of a feline, a canine and an equine. In an embodiment, the controlled release dosage form is administered to the subject parenterally, suitable examples of which are described elsewhere herein.

As noted elsewhere herein, several (i.e., multiple) divided doses may be administered daily, weekly, monthly or other suitable time intervals, or the dose may be proportionally reduced as indicated by the exigencies of the situation. Where a course of multiple doses is required or otherwise desired, it may be beneficial to administer the peptides, as herein disclosed, via more than one route. For example, it may be desirable to administer a first dose parenterally (e.g., via intramuscular, intravenous; subcutaneous, epidural, intra-articular, intraperitoneal, intracisternal or intrathecal routes of administration) to induce a rapid or acute therapeutic effect in a subject, followed by a subsequent (e.g., second, third, fourth, fifth, etc) dose administered enterally (e.g., orally or rectally), by inhalation or insufflation and/or topically (e.g., via transdermal or transmucosal routes of administration) to provide continuing availability of the active agent over an extended period subsequent to the acute phase of treatment. Alternatively, it may be desirable to administer a dose enterally (e.g., orally or rectally), followed by a subsequent (e.g., second, third, fourth, fifth, etc) dose administered parenterally (e.g., via intramuscular, intravenous; subcutaneous, epidural, intra-articular, intraperitoneal, intracisternal or intrathecal routes of administration), by inhalation or insufflation and/or topically (e.g., via transdermal or transmucosal routes of administration). Alternatively, it may be desirable to administer a dose topically (e.g., via transdermal or transmucosal routes of administration), followed by a subsequent (e.g., second, third, fourth, fifth, etc) dose administered parenterally (e.g., via intramuscular, intravenous; subcutaneous, epidural, intra-articular, intraperitoneal, intracisternal or intrathecal routes of administration), by inhalation or insufflation and/or enterally (e.g., orally or rectally).

It is also to be understood that, where multiple routes of administration are desired, any combination of two or more routes of administration may be used in accordance with the methods disclosed herein. Illustrative examples of suitable combinations include, but are not limited to, (in order of administration), (a) parenteral-enteral; (b) parenteral-topical; (c) parenteral-enteral-topical; (d) parenteral-topical-enteral; (e) enteral-parenteral; (f) enteral-topical; (g) enteral-topical-parenteral; (h) enteral-parenteral-topical; (i) topical-parenteral; (j) topical-enteral; (k) topical-parenteral-enteral; (1) topical-enteral-parenteral; (m) parenteral-enteral-topical-parenteral; (n) parenteral-enteral-topical-enteral; etc.

### Pharmaceutical Compositions

The peptides of formulae (I), (II), (III) and (IV), or pharmaceutically acceptable salts thereof, may be formulated for administration to a subject as a neat chemical. However, in certain embodiments, it may be preferable to formulate the peptides of formulae (I), (II), (III) and (IV), and pharmaceutically acceptable salts thereof, as a pharmaceutical composition, including veterinary compositions. Thus, in another aspect disclosed herein, there is provided a pharmaceutical composition comprising a peptide of formula (I), or a pharmaceutically acceptable salt thereof, as described herein, for use in the treatment of an inflammatory airway disease in a subject:

(I)
(SEQ ID NO: 1)
R¹-CRSVEGSCG-R²

wherein
R¹ is selected from the group consisting of YLRIVQ (SEQ ID NO:45), LRIVQ (SEQ ID NO:46), RIVQ (SEQ ID NO:47), IVQ, VQ, and Q, or R¹ is absent; and
R² is F (phenylalanine), or R² is absent.

In an embodiment, the peptide is selected from the group consisting of

(SEQ ID NO: 2)
YLRIVQCRSVEGSCGF,
(SEQ ID NO: 3)
LRIVQCRSVEGSCGF,
(SEQ ID NO: 4)
CRSVEGSCG
and
(SEQ ID NO: 5)
CRSVEGSCGF.

In an embodiment, the peptide is YLRIVQCRSVEGSCGF (SEQ ID NO:2). In an embodiment, the peptide is CRSVEGSCG (SEQ ID NO:4). In an embodiment, the peptide is CRSVEGSCGF (SEQ ID NO:5).

In another aspect disclosed herein, there is provided use of a peptide of formula (I), or a pharmaceutically acceptable salt thereof, as described herein, in the manufacture of a medicament for the treatment of an inflammatory airway disease in a subject:

(I)
(SEQ ID NO: 1)
R¹-CRSVEGSCG-R²

wherein
R¹ is selected from the group consisting of YLRIVQ (SEQ ID NO:45), LRIVQ (SEQ ID NO:46), RIVQ (SEQ ID NO:47), IVQ, VQ, and Q, or R¹ is absent; and
R² is F (phenylalanine), or R² is absent.

In an embodiment, wherein the peptide is selected from the group consisting of YLRIVQCRSVEGSCGF (SEQ ID NO:2), LRIVQCRSVEGSCGF (SEQ ID NO:3), CRSVEGSCG (SEQ ID NO:4) and CRSVEGSCGF (SEQ ID NO:5). In an embodiment, the peptide is YLRIVQCRSVEGSCGF (SEQ ID NO:2). In an embodiment, the peptide is CRSVEGSCG (SEQ ID NO:4). In an embodiment, the peptide is CRSVEGSCGF (SEQ ID NO:5).

In another aspect disclosed herein, there is provided a pharmaceutical composition comprising a peptide of formula (II), or a pharmaceutically acceptable salt thereof, for use in the treatment of an inflammatory airway disease in a subject:

(II)
(SEQ ID NO: 6)
R¹-CRRFVESSC-R²

wherein
R¹ is selected from the group consisting of YLRVMK (SEQ ID NO:48), LRVMK (SEQ ID NO:49), RVMK (SEQ ID NO:50), VMK, MK, and K, or R¹ is absent; and
R² is selected from the group consisting of A (alanine) and AF (alanine-phenylalanine), or R² is absent.

In an embodiment, the peptide is selected from the group consisting of

(SEQ ID NO: 7)
YLRVMKCRRFVESSCAF,
(SEQ ID NO: 8)
LRVMKCRRFVESSCAF,
(SEQ ID NO: 9)
CRRFVESSCAF
and
(SEQ ID NO: 10)
CRRFVESSCA.

In an embodiment, the peptide is YLRVMKCRRFVESSCAF (SEQ ID NO:7). In an embodiment, the peptide is CRRFVESSCAF (SEQ ID NO:9). In an embodiment, the peptide is CRRFVESSCA (SEQ ID NO:10).

In another aspect disclosed herein, there is provided a use of a peptide of formula (II), or a pharmaceutically acceptable salt thereof, in the manufacture of a medicament for the treatment of an inflammatory airway disease in a subject:

(II)
(SEQ ID NO: 6)
R¹-CRRFVESSC-R²

wherein
R¹ is selected from the group consisting of YLRVMK (SEQ ID NO:48), LRVMK (SEQ ID NO:49), RVMK (SEQ ID NO:50), VMK, MK, and K, or R¹ is absent; and
R² is selected from the group consisting of A (alanine) and AF (alanine-phenylalanine), or R² is absent.

In an embodiment, the peptide is selected from the group consisting of YLRVMKCRRFVESSCAF (SEQ ID NO:7), LRVMKCRRFVESSCAF (SEQ ID NO:8), CRRFVESSCAF (SEQ ID NO:9) and CRRFVESSCA (SEQ ID NO:10). In an embodiment, the peptide is YLRVMKCRRFVESSCAF (SEQ ID NO:7). In an embodiment, the peptide is CRRFVESSCAF (SEQ ID NO:9). In an embodiment, the peptide is CRRFVESSCA (SEQ ID NO:10).

In another aspect disclosed herein, there is provided a pharmaceutical composition comprising a peptide of formula (III), or a pharmaceutically acceptable salt thereof, for use in the treatment of an inflammatory airway disease in a subject:

(III)
(SEQ ID NO: 11)
R¹-C-R-X¹-X²-P-X³-X⁴-X⁵-X⁶-C-R²

wherein
X¹, X³, X⁵, and X⁶ is an amino acid residue selected from the group consisting of serine, alanine, valine, leucine, isoleucine and glycine;
X² is arginine or lysine;
X⁴ is glutamic acid or aspartic acid;
R¹ is selected from the group consisting of:
S,

(SEQ ID NO: 12)
HS,
(SEQ ID NO: 13)
GHS,
(SEQ ID NO: 14)
PGHS,
(SEQ ID NO: 15)
APGHS,
(SEQ ID NO: 16)
EAPGHS,
(SEQ ID NO: 17)
SEAPGHS,
(SEQ ID NO: 18)
SSEAPGHS,
(SEQ ID NO: 19)
PSSEAPGHS,
(SEQ ID NO: 20)
DPSSEAPGHS
and
(SEQ ID NO: 21)
IDPSSEAPGHS,

or R¹ is absent; and
R² is selected from the group consisting of
S,

(SEQ ID NO: 22)
SS,
(SEQ ID NO: 23)
SSK,
(SEQ ID NO: 24)
SSKF,
(SEQ ID NO: 25)
SSKFS,
(SEQ ID NO: 26)
SSKFSW,
(SEQ ID NO: 27)
SSKFSWD,
(SEQ ID NO: 28)
SSKFSWDE,
(SEQ ID NO: 29)
SSKFSWDEY,
(SEQ ID NO: 30)
SSKFSWDEYE,
(SEQ ID NO: 31)
SSKFSWDEYEQ,
(SEQ ID NO: 32)
SSKFSWDEYEQY,
(SEQ ID NO: 33)
SSKFSWDEYEQYK,
(SEQ ID NO: 34)
SSKFSWDEYEQYKK,
and
(SEQ ID NO: 35)
SSKFSWDEYEQYKKE,

or R² is absent.

In another aspect disclosed herein, there is provided use of a peptide of formula (III), or a pharmaceutically acceptable salt thereof, in the manufacture of a medicament for the treatment of an inflammatory airway disease in a subject:

R¹—C—R—X¹—X²—P—X³—X⁴—X⁵—X⁶—C—R² (III) (SEQ ID NO:11)
wherein
X¹, X³, X⁵, and X⁶ is an amino acid residue selected from the group consisting of serine, alanine, valine, leucine, isoleucine and glycine;
X² is arginine or lysine;
X⁴ is glutamic acid or aspartic acid;
R¹ is selected from the group consisting of:
S,

(SEQ ID NO: 12)
HS,
(SEQ ID NO: 13)
GHS,
(SEQ ID NO: 14)
PGHS,
(SEQ ID NO: 15)
APGHS,
(SEQ ID NO: 16)
EAPGHS,
(SEQ ID NO: 17)
SEAPGHS,
(SEQ ID NO: 18)
SSEAPGHS,
(SEQ ID NO: 19)
PSSEAPGHS,
(SEQ ID NO: 20)
DPSSEAPGHS
and
(SEQ ID NO: 21)
IDPSSEAPGHS,

or R¹ is absent; and
R² is selected from the group consisting of
S,

(SEQ ID NO: 22)
SS,
(SEQ ID NO: 23)
SSK,
(SEQ ID NO: 24)
SSKF,
(SEQ ID NO: 25)
SSKFS,
(SEQ ID NO: 26)
SSKFSW,
(SEQ ID NO: 27)
SSKFSWD,
(SEQ ID NO: 28)
SSKFSWDE,
(SEQ ID NO: 29)
SSKFSWDEY,
(SEQ ID NO: 30)
SSKFSWDEYE,
(SEQ ID NO: 31)
SSKFSWDEYEQ,
(SEQ ID NO: 32)
SSKFSWDEYEQY,
(SEQ ID NO: 33)
SSKFSWDEYEQYK,
(SEQ ID NO: 34)
SSKFSWDEYEQYKK,
and
(SEQ ID NO: 35)
SSKFSWDEYEQYKKE,

or R² is absent.

In an embodiment disclosed herein, the peptide of formula (III) has an amino acid sequence selected from the group consisting of:

(SEQ ID NO: 36)
SCRSRPVESSC;
(SEQ ID NO: 37)
CRSRPVESSC;
(SEQ ID NO: 38)
CRSRPVESSCS;
and
(SEQ ID NO: 39)
SCRSRPVESSCS.

In another aspect disclosed herein, there is provided a pharmaceutical composition comprising a peptide of formula (IV) or a pharmaceutically acceptable salt thereof, for use in the treatment of an inflammatory airway disease in a subject:

(IV)
(SEQ ID NO: 40)
R¹-C-R-I-X₁-X₂-X₃-X₄-N-C-R²

wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK (SEQ ID NO:51), LKLLK (SEQ ID NO:52), KLLK (SEQ ID NO:53), LLK, LK, K or R¹ is absent; and
R² is G (glycine), or R² is absent, or R² is a pharmaceutically acceptable carrier.

In another aspect disclosed herein, there is provided a use of a peptide of formula (IV) or a pharmaceutically acceptable salt thereof, in the manufacture of a medicament for the treatment of an inflammatory airway disease in a subject:

(IV)
(SEQ ID NO: 40)
R¹-C-R-I-X₁-X₂-X₃-X₄-N-C-R²

wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK (SEQ ID NO:51), LKLLK (SEQ ID NO:52), KLLK (SEQ ID NO:53), LLK, LK, K or R¹ is absent; and
R² is G (glycine), or R² is absent, or R² is a pharmaceutically acceptable carrier.

In an embodiment, the peptide of formula (IV) is selected from the group consisting of amino acid sequence CRIIHNNNC (SEQ ID NO:41), CRIIHNNNCG (SEQ ID NO:42), CRIVYDSNC (SEQ ID NO:43) and CRIVYDSNCG (SEQ ID NO:44).

As noted elsewhere herein, the peptides of formulae (I), (II), (III) and (IV), or pharmaceutically acceptable salts thereof, may be administered together, either sequentially or in combination (e.g., as an admixture), with one or more other active agents appropriate to the underlying condition to be treated. For example, the compositions disclosed herein may be formulated for administration together, either sequentially or in combination (e.g., as an admixture), with an inhaled corticosteroid typically employed for the treatment of asthma. Other suitable combination or adjunct therapies will be familiar to persons skilled in the art, the choice of which will depend on the underlying condition or symptom thereof.

In an embodiment, the composition further comprises a pharmaceutically acceptable carrier, excipient or diluent, as described elsewhere herein. In an embodiment, the composition is formulated for oral administration. In another embodiment, the composition is formulated for administration by inhalation or insufflation.

The peptides of formulae (I), (II), (III) and (IV), or pharmaceutically acceptable salts thereof, may suitably be prepared as pharmaceutical compositions and unit dosage forms to be employed as solids (e.g., tablets or filled capsules) or liquids (e.g., solutions, suspensions, emulsions, elixirs, or capsules filled with the same) for oral use, in the form of ointments, suppositories or enemas for rectal administration, in the form of sterile injectable solutions for parenteral use (e.g., intramuscular, subcutaneous, intravenous, epidural, intra-articular and intrathecal administration); or in the form of ointments, lotions, creams, gels, patches, sublingual strips or films, and the like for parenteral (e.g., topical, buccal, sublingual, vaginal) administration. In an embodiment, the peptides of formulae (I), (II), (III) and (IV), or pharmaceutically acceptable salts thereof, are formulated for topical (e.g., transdermal) delivery. Suitable transdermal delivery systems will be familiar to persons skilled in the art, illustrative examples of which are described by Prausnitz and Langer (2008; Nature Biotechnol. 26(11):1261-1268), the contents of which are incorporated herein by reference. In another embodiment, the peptides of formulae (I), (II), (III) and (IV), or pharmaceutically acceptable salts thereof, are formulated for sublingual or buccal delivery. Suitable sublingual and buccal delivery systems will be familiar to persons skilled in the art, illustrative examples of which include dissolvable strips or films, as described by Bala et al. (2013; Int. J. Pharm. Investig. 3(2):67-76), the contents of which are incorporated herein by reference.

Suitable pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed. The peptides of formulae (I), (II), (III) and (IV), or pharmaceutically acceptable salts thereof, as described herein, can be formulated for administration in a wide variety of enteral, topical and/or parenteral dosage forms. Suitable dosage forms may comprise, as the active component, either a peptide of formula (I), a peptide of formula (II), a peptide of formula (III), a peptide of formula (IV), pharmaceutically acceptable salts thereof, or combinations of any of the foregoing, as herein described.

In an embodiment, the composition is formulated for oral administration to a human. In another embodiment, the composition is formulated for oral administration to a non-human subject. In yet another embodiment, the composition is formulated for oral administration to a non-human subject selected from the group consisting of a feline, a canine and an equine.

In another embodiment, the composition is formulated for parenteral administration to a human. In another embodiment, the composition is formulated for parenteral administration to a non-human subject. In yet another embodiment, the composition is formulated for parenteral administration to a non-human subject selected from the group consisting of a feline, a canine and an equine. In an embodiment, the parenteral administration is subcutaneous administration.

In another embodiment, the composition is formulated for topical administration to a human. In another embodiment, the composition is formulated for topical administration to a non-human subject. In yet another embodiment, the composition is formulated for topical administration to a non-human subject selected from the group consisting of a feline, a canine and an equine. In an embodiment, the topical administration is transdermal.

In another embodiment, the composition is formulated for administration to a human by inhalation or insufflation. In another embodiment, the composition is formulated for administration to a non-human subject by inhalation or insufflation. In yet another embodiment, the composition is formulated for administration by inhalation or insufflation to a non-human subject selected from the group consisting of a feline, a canine and an equine.

In another embodiment, the composition is formulated as a controlled release dosage form to be administered to a human. In another embodiment, the composition is formulated as a controlled release dosage form to be administered to a non-human subject. In yet another embodiment, the composition is formulated as a controlled release dosage form to be administered to a non-human subject selected from the group consisting of a feline, a canine and an equine. Illustrative examples of suitable controlled release dosage forms are described elsewhere herein.

For preparing pharmaceutical compositions of the peptides of formulae (I), (II), (III) and (IV), or pharmaceutically acceptable salts thereof, pharmaceutically acceptable carriers can be either solid or liquid. Illustrative examples of solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material. In powders, the carrier may be a finely divided solid which is in a mixture with the finely divided active component. In tablets, the active component may be mixed with the carrier having the necessary binding capacity in suitable proportions and compacted in the shape and size desired.

In some embodiments, the powders and tablets contain from five or ten to about seventy percent of the active compound. Illustrative examples of suitable carriers include magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material, providing a capsule in which the active component, with or without carriers, is surrounded by a carrier. Similarly, cachets and lozenges are also envisaged herein. Tablets, powders, capsules, pills, cachets, and lozenges can be used as solid forms suitable for oral administration.

For preparing suppositories, a low melting wax, such as admixture of fatty acid glycerides or cocoa butter, is first melted and the active component is dispersed homogeneously therein, as by stirring. The molten homogenous mixture is then poured into convenient sized molds, allowed to cool, and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution.

The peptides of formulae (I), (II), (III) and (IV), or pharmaceutically acceptable salts thereof, as described herein, may be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active compound(s) may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also contemplated herein are solid form preparations which are intended to be converted, shortly before use, to liquid form preparations for oral administration. Such liquid forms include solutions, suspensions, and emulsions. These preparations may contain, in addition to the active component, colorants, flavours, stabilizers, buffers, artificial and natural sweeteners, dispersants, thickeners, solubilizing agents, and the like.

For topical administration to the epidermis, the peptides of formulae (I), (II) or (III), or pharmaceutically acceptable salts thereof, as described herein, may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or colouring agents.

Formulations suitable for topical administration in the mouth include lozenges comprising active agent in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multidose form. In the latter case of a dropper or pipette, this may be achieved by the patient administering an appropriate, predetermined volume of the solution or suspension. In the case of a spray, this may be achieved for example by means of a metering atomizing spray pump. To improve nasal delivery and retention the peptides used in the invention may be encapsulated with cyclodextrins, or formulated with their agents expected to enhance delivery and retention in the nasal mucosa.

Administration to the airways may also be achieved by means of an aerosol formulation in which the active ingredient is provided in a pressurised pack with a suitable propellant such as a chlorofluorocarbon (CFC) for example, dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively, or in addition, the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently, the powder carrier will form a gel in the nasal cavity. The powder composition may be presented in unit dose form for example in capsules or cartridges of, e.g., gelatin, or blister packs from which the powder may be administered by means of an inhaler.

In formulations intended for administration to the airways, including intranasal formulations, the peptide will generally have a small particle size for example of the order of 1 to 10 microns or less. Such a particle size may be obtained by means known in the art, for example by micronization.

When desired, formulations adapted to give controlled or sustained release of the active ingredient may be employed, as described elsewhere herein.

In an embodiment, the pharmaceutical preparations, as herein described, are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

In an embodiment, the compositions disclosed herein are formulated for oral administration to a human. In yet another embodiment, the compositions disclosed herein are formulated for oral administration to a non-human. In a further embodiment, the compositions disclosed herein are formulated for oral administration to a non-human selected from the group consisting of a feline, a canine and an equine.

In an embodiment, the compositions disclosed herein are formulated for administration to a human by inhalation or insufflation. In yet another embodiment, the compositions disclosed herein are formulated for administration to a non-human by inhalation or insufflation. In a further embodiment, the compositions disclosed herein are formulated for administration by inhalation or insufflation to a non-human selected from the group consisting of a feline, a canine and an equine.

In another embodiment, the peptide of formula (I), or a pharmaceutically acceptable salt thereof, as disclosed herein, is formulated for oral administration to a human subject. In another embodiment, the peptide of formula (I), or a pharmaceutically acceptable salt thereof, as disclosed herein, is formulated for oral administration to a non-human subject. In yet another embodiment, the peptide of formula (I), or a pharmaceutically acceptable salt thereof, as disclosed herein, is formulated for oral administration to a non-human subject selected from the group consisting of a feline, a canine and an equine.

In another embodiment, the peptide of formula (I), or a pharmaceutically acceptable salt thereof, as disclosed herein, is formulated for topical administration to a human subject. In yet another embodiment, the peptide of formula (I), or a pharmaceutically acceptable salt thereof, as disclosed herein, is formulated for topical administration to a non-human subject. In another embodiment, the peptide of formula (I), or a pharmaceutically acceptable salt thereof, as disclosed herein, is formulated for topical administration to a non-human subject selected from the group consisting of a feline, a canine and an equine. In an embodiment, the topical administration is transdermal.

In another embodiment, the peptide of formula (I), or a pharmaceutically acceptable salt thereof, as disclosed herein, is formulated for administration to a human subject by inhalation or insufflation. In yet another embodiment, the peptide of formula (I), or a pharmaceutically acceptable salt thereof, as disclosed herein, is formulated for administration to a non-human subject by inhalation or insufflation. In another embodiment, the peptide of formula (I), or a pharmaceutically acceptable salt thereof, as disclosed herein, is formulated for administration by inhalation or insufflation to a non-human subject selected from the group consisting of a feline, a canine and an equine.

In another embodiment, the peptide of formula (I), or a pharmaceutically acceptable salt thereof, as disclosed herein, is formulated for administration to a human subject as a controlled release dosage form. In yet another embodiment, the peptide of formula (I), or a pharmaceutically acceptable salt thereof, as disclosed herein, is formulated for administration to a non-human subject as a controlled release dosage form. In another embodiment, the peptide of formula (I), or a pharmaceutically acceptable salt thereof, as disclosed herein, is formulated for administration to a non-human subject as a controlled release dosage form, wherein the non-human subject is selected from the group consisting of a feline, a canine and an equine. In an embodiment, the controlled release dosage form is formulated for parenteral administration.

In another embodiment, the peptide of formula (II), or a pharmaceutically acceptable salt thereof, as disclosed herein, are formulated for oral administration to a non-human subject. In yet another embodiment, the peptide of formula (II), or a pharmaceutically acceptable salt thereof, as disclosed herein, is formulated for oral administration to a non-human subject selected from the group consisting of a feline, a canine and an equine.

In another embodiment, the peptide of formula (II), or a pharmaceutically acceptable salt thereof, as disclosed herein, is formulated for topical administration to a non-human subject. In yet another embodiment, the peptide of formula (II), or a pharmaceutically acceptable salt thereof, as disclosed herein, is formulated for topical administration to a non-human subject selected from the group consisting of a feline, a canine and an equine. In an embodiment, the topical administration is transdermal.

In another embodiment, the peptide of formula (II), or a pharmaceutically acceptable salt thereof, as disclosed herein, is formulated for administration to a human subject by inhalation or insufflation. In yet another embodiment, the peptide of formula (II), or a pharmaceutically acceptable salt thereof, as disclosed herein, is formulated for administration to a non-human subject by inhalation or insufflation. In another embodiment, the peptide of formula (II), or a pharmaceutically acceptable salt thereof, as disclosed herein, is formulated for administration by inhalation or insufflation to a non-human subject selected from the group consisting of a feline, a canine and an equine.

In another embodiment, the peptide of formula (II), or a pharmaceutically acceptable salt thereof, as disclosed herein, is formulated for administration to a human subject as a controlled release dosage form. In yet another embodiment, the peptide of formula (II), or a pharmaceutically acceptable salt thereof, as disclosed herein, is formulated for administration to a non-human subject as a controlled release dosage form. In another embodiment, the peptide of formula (II), or a pharmaceutically acceptable salt thereof, as disclosed herein, is formulated for administration to a non-human subject as a controlled release dosage form, wherein the non-human subject is selected from the group consisting of a feline, a canine and an equine. In an embodiment, the controlled release dosage form is formulated for parenteral administration.

In another embodiment, the peptide of formula (III), or a pharmaceutically acceptable salt thereof, as disclosed herein, are formulated for oral administration to a non-human subject. In yet another embodiment, the peptide of formula (III), or a pharmaceutically acceptable salt thereof, as disclosed herein, is formulated for oral administration to a non-human subject selected from the group consisting of a feline, a canine and an equine.

In another embodiment, the peptide of formula (III), or a pharmaceutically acceptable salt thereof, as disclosed herein, is formulated for topical administration to a non-human subject. In yet another embodiment, the peptide of formula (III), or a pharmaceutically acceptable salt thereof, as disclosed herein, is formulated for topical administration to a non-human subject selected from the group consisting of a feline, a canine and an equine. In an embodiment, the topical administration is transdermal.

In another embodiment, the peptide of formula (III), or a pharmaceutically acceptable salt thereof, as disclosed herein, is formulated for administration to a human subject by inhalation or insufflation. In yet another embodiment, the peptide of formula (III), or a pharmaceutically acceptable salt thereof, as disclosed herein, is formulated for administration to a non-human subject by inhalation or insufflation. In another embodiment, the peptide of formula (III), or a pharmaceutically acceptable salt thereof, as disclosed herein, is formulated for administration by inhalation or insufflation to a non-human subject selected from the group consisting of a feline, a canine and an equine.

In another embodiment, the peptide of formula (III), or a pharmaceutically acceptable salt thereof, as disclosed herein, is formulated for administration to a human subject as a controlled release dosage form. In yet another embodiment, the peptide of formula (III), or a pharmaceutically acceptable salt thereof, as disclosed herein, is formulated for administration to a non-human subject as a controlled release dosage form. In another embodiment, the peptide of formula (III), or a pharmaceutically acceptable salt thereof, as disclosed herein, is formulated for administration to a non-human subject as a controlled release dosage form, wherein the non-human subject is selected from the group consisting of a feline, a canine and an equine. In an embodiment, the controlled release dosage form is formulated for parenteral administration.

In another embodiment, the peptide of formula (IV), or a pharmaceutically acceptable salt thereof, as disclosed herein, are formulated for oral administration to a non-human subject. In yet another embodiment, the peptide of formula (IV), or a pharmaceutically acceptable salt thereof, as disclosed herein, is formulated for oral administration to a non-human subject selected from the group consisting of a feline, a canine and an equine.

In another embodiment, the peptide of formula (IV), or a pharmaceutically acceptable salt thereof, as disclosed herein, is formulated for topical administration to a non-human subject. In yet another embodiment, the peptide of formula (IV), or a pharmaceutically acceptable salt thereof, as disclosed herein, is formulated for topical administration to a non-human subject selected from the group consisting of a feline, a canine and an equine. In an embodiment, the topical administration is transdermal.

In another embodiment, the peptide of formula (IV), or a pharmaceutically acceptable salt thereof, as disclosed herein, is formulated for administration to a human subject by inhalation or insufflation. In yet another embodiment, the peptide of formula (IV), or a pharmaceutically acceptable salt thereof, as disclosed herein, is formulated for administration to a non-human subject by inhalation or insufflation. In another embodiment, the peptide of formula (IV), or a pharmaceutically acceptable salt thereof, as disclosed herein, is formulated for administration by inhalation or insufflation to a non-human subject selected from the group consisting of a feline, a canine and an equine.

In another embodiment, the peptide of formula (IV), or a pharmaceutically acceptable salt thereof, as disclosed herein, is formulated for administration to a human subject as a controlled release dosage form. In yet another embodiment, the peptide of formula (IV), or a pharmaceutically acceptable salt thereof, as disclosed herein, is formulated for administration to a non-human subject as a controlled release dosage form. In another embodiment, the peptide of formula (IV), or a pharmaceutically acceptable salt thereof, as disclosed herein, is formulated for administration to a non-human subject as a controlled release dosage form, wherein the non-human subject is selected from the group consisting of a feline, a canine and an equine. In an embodiment, the controlled release dosage form is formulated for parenteral administration.

In another embodiment, the peptide of SEQ ID NO:2, or a pharmaceutically acceptable salt thereof, is formulated for oral administration to a human. In another embodiment, the peptide of SEQ ID NO:2, or a pharmaceutically acceptable salt thereof, is formulated for oral administration to a non-human. subject. In yet another embodiment, the peptide of SEQ ID NO:2, or a pharmaceutically acceptable salt thereof, is formulated for oral administration to a non-human subject selected from the group consisting of a feline, a canine and an equine.

In another embodiment, the peptide of SEQ ID NO:2, or a pharmaceutically acceptable salt thereof, as disclosed herein, is formulated for topical administration to a human subject. In yet another embodiment, the peptide of SEQ ID NO:2, or a pharmaceutically acceptable salt thereof, as disclosed herein, is formulated for topical administration to a non-human subject. In another embodiment, the peptide of SEQ ID NO:2, or a pharmaceutically acceptable salt thereof, as disclosed herein, is formulated for topical administration to a non-human subject selected from the group consisting of a feline, a canine and an equine. In an embodiment, the topical administration is transdermal.

In another embodiment, the peptide of SEQ ID NO:2, or a pharmaceutically acceptable salt thereof, as disclosed herein, is formulated for administration to a human subject by inhalation or insufflation. In yet another embodiment, the peptide of SEQ ID NO:2, or a pharmaceutically acceptable salt thereof, as disclosed herein, is formulated for administration to a non-human subject by inhalation or insufflation. In another embodiment, the peptide of SEQ ID NO:2, or a pharmaceutically acceptable salt thereof, as disclosed herein, is formulated for administration by inhalation or insufflation to a non-human subject selected from the group consisting of a feline, a canine and an equine.

In another embodiment, the peptide of SEQ ID NO:2, or a pharmaceutically acceptable salt thereof, as disclosed herein, is formulated for administration to a human subject as a controlled release dosage form. In yet another embodiment, the peptide of SEQ ID NO:2, or a pharmaceutically acceptable salt thereof, as disclosed herein, is formulated for administration to a non-human subject as a controlled release dosage form. In another embodiment, the peptide of SEQ ID NO:2, or a pharmaceutically acceptable salt thereof, as disclosed herein, is formulated for administration to a non-human subject as a controlled release dosage form, wherein the non-human subject is selected from the group consisting of a feline, a canine and an equine. In an embodiment, the controlled release dosage form is formulated for parenteral administration.

In another embodiment, the peptide of SEQ ID NO:7, or a pharmaceutically acceptable salt thereof, is formulated for oral administration to a non-human. subject. In yet another embodiment, the peptide of SEQ ID NO:7, or a pharmaceutically acceptable salt thereof, is formulated for oral administration to a non-human subject selected from the group consisting of a feline, a canine and an equine.

In another embodiment, the peptide of SEQ ID NO:7, or a pharmaceutically acceptable salt thereof, is formulated for topical administration to a non-human. subject. In yet another embodiment, the peptide of SEQ ID NO:7, or a pharmaceutically acceptable salt thereof, is formulated for topical administration to a non-human subject selected from the group consisting of a feline, a canine and an equine. In an embodiment, the topical administration is transdermal.

In another embodiment, the peptide of SEQ ID NO:7, or a pharmaceutically acceptable salt thereof, as disclosed herein, is formulated for administration to a human subject by inhalation or insufflation. In yet another embodiment, the peptide of SEQ ID NO:7, or a pharmaceutically acceptable salt thereof, as disclosed herein, is formulated for administration to a non-human subject by inhalation or insufflation. In another embodiment, the peptide of SEQ ID NO:7, or a pharmaceutically acceptable salt thereof, as disclosed herein, is formulated for administration by inhalation or insufflation to a non-human subject selected from the group consisting of a feline, a canine and an equine.

In another embodiment, the peptide of SEQ ID NO:7, or a pharmaceutically acceptable salt thereof, as disclosed herein, is formulated for administration to a human subject as a controlled release dosage form. In yet another embodiment, the peptide of SEQ ID NO:7, or a pharmaceutically acceptable salt thereof, as disclosed herein, is formulated for administration to a non-human subject as a controlled release dosage form. In another embodiment, the peptide of SEQ ID NO:7, or a pharmaceutically acceptable salt thereof, as disclosed herein, is formulated for administration to a non-human subject as a controlled release dosage form, wherein the non-human subject is selected from the group consisting of a feline, a canine and an equine. In an embodiment, the controlled release dosage form is formulated for parenteral administration.

In another embodiment, the peptide of SEQ ID NO:36, or a pharmaceutically acceptable salt thereof, is formulated for oral administration to a non-human. subject. In yet another embodiment, the peptide of SEQ ID NO:36, or a pharmaceutically acceptable salt thereof, is formulated for oral administration to a non-human subject selected from the group consisting of a feline, a canine and an equine.

In another embodiment, the peptide of SEQ ID NO:36, or a pharmaceutically acceptable salt thereof, is formulated for topical administration to a non-human. subject. In yet another embodiment, the peptide of SEQ ID NO:36, or a pharmaceutically acceptable salt thereof, is formulated for topical administration to a non-human subject selected from the group consisting of a feline, a canine and an equine. In an embodiment, the topical administration is transdermal.

In another embodiment, the peptide of SEQ ID NO:36, or a pharmaceutically acceptable salt thereof, as disclosed herein, is formulated for administration to a human subject by inhalation or insufflation. In yet another embodiment, the peptide of SEQ ID NO:36, or a pharmaceutically acceptable salt thereof, as disclosed herein, is formulated for administration to a non-human subject by inhalation or insufflation. In another embodiment, the peptide of SEQ ID NO:36, or a pharmaceutically acceptable salt thereof, as disclosed herein, is formulated for administration by inhalation or insufflation to a non-human subject selected from the group consisting of a feline, a canine and an equine.

In another embodiment, the peptide of SEQ ID NO:36, or a pharmaceutically acceptable salt thereof, as disclosed herein, is formulated for administration to a human subject as a controlled release dosage form. In yet another embodiment, the peptide of SEQ ID NO:36, or a pharmaceutically acceptable salt thereof, as disclosed herein, is formulated for administration to a non-human subject as a controlled release dosage form. In another embodiment, the peptide of SEQ ID NO:36, or a pharmaceutically acceptable salt thereof, as disclosed herein, is formulated for administration to a non-human subject as a controlled release dosage form, wherein the non-human subject is selected from the group consisting of a feline, a canine and an equine. In an embodiment, the controlled release dosage form is formulated for parenteral administration.

In another embodiment, the peptide of SEQ ID NO:41, or a pharmaceutically acceptable salt thereof, is formulated for oral administration to a human. In another embodiment, the peptide of SEQ ID NO:41, or a pharmaceutically acceptable salt thereof, is formulated for oral administration to a non-human. subject. In yet another embodiment, the peptide of SEQ ID NO:41, or a pharmaceutically acceptable salt thereof, is formulated for oral administration to a non-human subject selected from the group consisting of a feline, a canine and an equine.

In another embodiment, the peptide of SEQ ID NO:41, or a pharmaceutically acceptable salt thereof, as disclosed herein, is formulated for topical administration to a human subject. In yet another embodiment, the peptide of SEQ ID NO:41, or a pharmaceutically acceptable salt thereof, as disclosed herein, is formulated for topical administration to a non-human subject. In another embodiment, the peptide of SEQ ID NO:41, or a pharmaceutically acceptable salt thereof, as disclosed herein, is formulated for topical administration to a non-human subject selected from the group consisting of a feline, a canine and an equine. In an embodiment, the topical administration is transdermal.

In another embodiment, the peptide of SEQ ID NO:41, or a pharmaceutically acceptable salt thereof, as disclosed herein, is formulated for administration to a human subject by inhalation or insufflation. In yet another embodiment, the peptide of SEQ ID NO:41, or a pharmaceutically acceptable salt thereof, as disclosed herein, is formulated for administration to a non-human subject by inhalation or insufflation. In another embodiment, the peptide of SEQ ID NO:41, or a pharmaceutically acceptable salt thereof, as disclosed herein, is formulated for administration by inhalation or insufflation to a non-human subject selected from the group consisting of a feline, a canine and an equine.

In another embodiment, the peptide of SEQ ID NO:41, or a pharmaceutically acceptable salt thereof, as disclosed herein, is formulated for administration to a human subject as a controlled release dosage form. In yet another embodiment, the peptide of SEQ ID NO:41, or a pharmaceutically acceptable salt thereof, as disclosed herein, is formulated for administration to a non-human subject as a controlled release dosage form. In another embodiment, the peptide of SEQ ID NO:41, or a pharmaceutically acceptable salt thereof, as disclosed herein, is formulated for administration to a non-human subject as a controlled release dosage form, wherein the non-human subject is selected from the group consisting of a feline, a canine and an equine. In an embodiment, the controlled release dosage form is formulated for parenteral administration.

As noted elsewhere herein, several (i.e., multiple) divided doses may be administered daily, weekly, monthly or other suitable time intervals, or the dose may be proportionally reduced as indicated by the exigencies of the situation. Where a course of multiple doses is required or otherwise desired, the compositions disclosed herein can be suitably formulated for administration via said multiple routes. For example, it may be desirable to administer a first dose parenterally (e.g., intramuscular, intravenously; subcutaneously, etc) to induce a rapid or otherwise acute therapeutic effect in a subject, followed by a subsequent (e.g., second, third, fourth, fifth, etc) dose administered non-parenterally (e.g., enterally and/or topically) to provide continuing availability of the active agent over an extended period subsequent to the acute phase of treatment. Thus, in an embodiment, the peptides and compositions, as disclosed herein, are formulated for parenteral administration to the subject as a first dose (i.e., as a parenteral dosage form) and formulated for non-parenteral administration to the subject after the first dose (e.g., as an enteral and/or topical dosage form). In an embodiment, the parental administration is selected from the group consisting of intramuscular, subcutaneous and intravenous. In a further embodiment, the parental administration is subcutaneous.

In another embodiment, the enteral administration is oral administration. Thus, in an embodiment, the peptides and compositions, as disclosed herein, are formulated for parenteral administration to the subject as a first dose and formulated for oral administration to the subject after the first dose (i.e., as an oral dosage form).

In another embodiment, the enteral administration is topical administration. Thus, in an embodiment, the peptides and compositions, as disclosed herein, are formulated for parenteral administration to the subject as a first dose and formulated for topical administration to the subject after the first dose (i.e., as an oral dosage form). In an embodiment, the topical administration is transdermal administration.

In another embodiment, it may be desirable to administer a first dose parenterally (e.g., intramuscular, intravenously; subcutaneously, etc) to induce a rapid or otherwise acute therapeutic effect in a subject, followed by a subsequent (e.g., second, third, fourth, fifth, etc) administration of a controlled release dosage form, as described elsewhere herein, to provide a controlled release of the active agent over an extended period subsequent to the acute phase of treatment. Thus, in another embodiment, the peptides and compositions, as disclosed herein, are formulated for parenteral administration to the subject as a first dose and formulated as a controlled release dosage form to be administered to the subject after the first dose. In an embodiment, the controlled release dosage form is formulated for parental administration.

It may also be desirable to administer a first dose enterally (e.g., orally or rectally), followed by a subsequent (e.g., second, third, fourth, fifth, etc) dose administered topically (e.g., transdermally). Thus, in an embodiment, the peptides and compositions, as disclosed herein, are formulated for enteral administration to the subject as a first dose (i.e., as an enteral dosage form; oral or rectal) and formulated for topical administration to the subject after the first dose (e.g., as a transdermal or transmucosal dosage form). In another embodiment, the peptides and compositions, as disclosed herein, are formulated for topical administration selected from the group consisting of transdermal and transmucosal administration. In a further embodiment, the peptides and compositions, as disclosed herein, are formulated for transdermal administration.

In yet another embodiment, it may be desirable to administer the peptides or compositions, as disclosed herein, enterally (e.g., orally or rectally) as a first dose, followed by a subsequent (e.g., second, third, fourth, fifth, etc) dose as a controlled release dosage form, as described elsewhere herein. Thus, in an embodiment, the peptides and compositions, as disclosed herein, are formulated for administration as a first dose enterally and formulated for administration as a controlled release dosage form, wherein the controlled release dosage form is formulated for administration subsequent to the first dose. In an embodiment, the enteral dose is formulated for oral administration. In another embodiment, the controlled release dosage form is formulated for parenteral administration.

In an embodiment, it may be desirable to administer the peptides or compositions, as disclosed herein, topically (e.g., orally or rectally) as a first dose, followed by a subsequent (e.g., second, third, fourth, fifth, etc) dose as a controlled release dosage form, as described elsewhere herein. Thus, in an embodiment, the peptides and compositions, as disclosed herein, are formulated for topical administration as a first dose and formulated for administration as a controlled release dosage form, wherein the controlled release dosage form is formulated for administration subsequent to the first topical dose. In an embodiment, the topical dose is formulated for transdermal administration. In another embodiment, the controlled release dosage form is formulated for parenteral administration.

The invention will now be described with reference to the following Examples which illustrate some preferred aspects of the present invention. However, it is to be understood that the particularity of the following description of the invention is not to supersede the generality of the preceding description of the invention.

### EXAMPLES

### Example 1: Effect of Peptides on Airway Inflammation in an Experimental Animal Model of Cigarette Smoke-Induced, Chronic Obstructive Pulmonary Disease (COPD)

### Animal Model of COPD

24 female c57Bl/6 mice were divided into 3 groups as outlined in Table 2, below. Mice in group 1 were exposed to room air for 2 weeks and weighed 3× per week. Mice in groups 2 and 3 were exposed to cigarette smoke (CS) twice daily, 5 days per week for 2 weeks and weighed 5× per week. The cigarette smoke was delivered to mice using a custom designed and purpose-built nose-only, directed-flow inhalation and smoke-exposure system (CH Technologies, Westwood, NJ, USA) with an air flow rate of 2.5 L/min, housed in a fume and laminar flow hood. To test the therapeutic potential of LAT8881, mice in group 3 were treated with LAT8881 (20 mg/kg, +2 days) via intranasal instillation 2 hours prior to the cigarette smoke exposure. Mice were then culled at the end of the 2^{nd} week period of smoke exposure by administering an overdose of sodium pentobarbitone (up to 325 mg/kg mice) intraperitoneally and samples collected.

TABLE 2

| | # mice per | | | |
|---|---|---|---|---|
| Group | group | Groups | Treatments | Time point |
| 1 | 8 | Air | N/A | Day 11 |
| 2 | 8 | CS | N/A | Day 11 |
| 3 | 8 | CS | LAT8881 (i.n. 20 mg/kg) | Day 11 |

### BALF Collection, Processing and Staining

The multi-lobes of the lungs were tied off at the right bronchi with a string, and the individual lobes were removed and collected for further molecular analysis. The intact left lung lobe was used to collect bronchoalveolar lavage fluid (BALF). The BALF was collected by lavaging lungs (after death) with two 500 μL aliquots of PBS at room temperature.

The BALF was processed by centrifuging it at 132×g for 5 mins at 4° C.; the resultant supernatant was collected and stored at −80° C. for further assessment. The cell pellet was resuspended with red blood cell (RBC) lysis buffer (0.15M ammonium chloride (Sigma Aldrich, Castle Hill, NSW, Australia), 0.01M sodium bicarbonate (Sigma Aldrich, Castle Hill, NSW, Australia), 0.001M EDTA (Sigma Aldrich, Castle Hill, NSW, Australia) for 5 min at 4° C. 1 ml of PBS was added to stop the activity of the red blood cell lysis buffer The solution was centrifuged at 132×g for 5 mins at 4° C.; this time the resultant supernatant was discarded and the cell pellet was resuspended with 160 μl of PBS. The total leukocyte count was calculated using the trypan blue exclusion method using a haemocytometer. The remaining sample was cytospun at 300 rpm for 10 mins on to clean microscope slides using the cytospin (Shandon, Cheshire, England). The slides were then allowed to dry overnight before being stained for histological assessment.

To determine the proportion of individual inflammatory cells, macrophages, neutrophils, lymphocytes in the BALF, the cells that were cytospun on slides were stained with May-Grunwald Giesma stain to allow for enumeration of individual cell types. The slides were immersed in May-Grunwald Stain (Sigma Aldrich, Castle Hill, NSW, Australia) for 5 mins, followed by one min wash with distilled water. The slides were then counterstained with Giemsa reagent (Australian Scientific Proprietary Limited, NSW Australia) for 20 mins and washed with distilled water twice for 5 mins each. The slides were left to dry overnight and were cover-slipped using Entellan® mounting media (Merck Millipore, Bayswater, VIC, Australia).

The enumeration of the inflammatory cells was conducted by counting a total of 200 cells under the light microscope (40× magnification), and the cells were differentiated based on their morphology.

### Lung Processing for Histology

The left lung was then perfused by gently injecting 0.9% sodium chloride solution (25 G needle) through the apex of the right ventricle of the heart at constant pressure until the lung was inflated and changed its colour to white/pink. The left lung was then inflated and fixed with 10% neutral buffered formalin (Lonza Australia Proprietary Limited, Waverley Vic, Australia) via the trachea. The left lung was then excised and stored in formalin for a minimum of 24 hours to allow the tissue fixation, which is essential to preserve the cell and tissue morphology. The fixed-left lung was then transferred to 10% ethanol (AnalaR NORMAPUR® ACS, Avantor) in Phosphate-buffered saline (PBS) (ThermoFisher Scientific, Grand Island, New York).

The lungs were processed using Leica HistoCore PEARL tissue processor and the processed lungs were paraffin embedded and sectioned using the Leica RM2245 semi-automated microtome. The sections (3.5 μm thickness) were mounted on microscope slides and then stained with haematoxylin and eosin for further histological analysis.

### Statistical Analysis

The data are presented as mean±standard error of the mean (SEM). To test the normality, Normal Gaussian distribution was assessed. If the data were normally distributed, t-tests was performed (parametric test) to compare two groups and one-way ANOVA was performed for more than two groups. If the data were not normally distributed, Kruskal Wallis test was performed. The exclusion of outliers (Grubbs and/or ROUT test), test for Gaussian distribution (D'Agostino-Pearson omnibus normality test, Shapiro-Wilk normality test and Kolmogorov-Smirnov normality test) and the like were performed using GraphPad Prism V.9.0.2 software (San Diego, California, USA). The differences were considered significant if <0.05.

### Example 2: Effect of Peptides on Airway Inflammation and Lung Function in an Experimental Animal Model of COPD

32 female c57Bl/6 mice were divided into 4 groups as outlined in Table 3, below. Mice in Group 1 were exposed to room air for 4 weeks. Mice in groups 2, 3 and 4 were exposed to cigarette smoke (CS) twice daily, 5 days per week for 8 weeks. The cigarette smoke was delivered to mice using a custom designed and purpose-built nose-only, directed-flow inhalation and smoke-exposure system (CH Technologies, Westwood, NJ, USA) with an air flow rate of 2.5 L/min, housed in a fume and laminar flow hood. To test the therapeutic potential of LAT8881, mice in group 3 were treated with LAT8881 (20 mg/kg) every second day from day +2 via intranasal instillation 2 hours prior to the cigarette smoke exposure. Mice in group 4 were treated with LAT8881 (20 mg/kg) every second day from day +42 via intranasal instillation 2 hours prior to the cigarette smoke exposure. Mice were then culled at the end of the 8^{nd} week period of smoke exposure by administering an overdose of sodium pentobarbitone (up to 325 mg/kg mice) intraperitoneally and samples collected.

TABLE 3

| | # mice per | | | |
|---|---|---|---|---|
| Group | group | Groups | Treatments | Time point |
| 1 | 8 | Air | N/A | Day 56 |
| 2 | 8 | CS | N/A | Day 56 |
| 3 | 8 | CS | LAT8881 (i.n. 20 mg/kg) | Day 56 |
| | | | from day +2 | |
| 4 | 8 | CS | LAT8881 (i.n. 20 mg/kg) | Day 56 |
| | | | from day +42 | |

### Lung Function

Mice were anaesthetised with ketamine (100 mg/kg) and xylazine (10 mg/kg). They were then cannulated (tracheostomy with ligation). The flexiVent apparatus (FlexiVent and FlexiVent with Forced Expiration Volume Extension [FEV] (Scireq); Montreal, Quebec, Canada) was used to assess hysteresis, transpulmonary resistance and compliance, tissue damping and airway-specific resistance at baseline (by using a tidal volume of 8 mL/kg at a respiratory rate of 450 breaths/min). The FEV extension was used to assess peak expiratory flow, forced vital capacity, forced expired volumes and flows. This combination of anaesthesia and ventilation is common and recommended by the manufacturer. Maximal pressure-volume loops were used to calculate hysteresis. Transpulmonary resistance and dynamic compliance were assessed by using the snapshot perturbation function. The forced oscillation perturbation was then used to determine the airway's (Newtonian) resistance and tissue damping. For all perturbations, a coefficient of determination of 0.95 was the minimum allowable for an acceptable measurement. Each perturbation was conducted 3 times per animal, and the average was calculated, with a minimum ventilation period of 20 seconds allowed between each perturbation (see Beckett et al., J. Allergy and Cin. Immunol., 2013, 131(3):752-762).

### BALF Collection, Processing and Staining

The multi-lobes of the lungs were tied off at the right bronchi, and the individual lobes were removed and collected for further molecular analysis. The intact left lung lobe was used to collect bronchoalveolar lavage fluid (BALF). The BALF was collected by lavaging lungs with two 500 μL aliquots of PBS at room temperature.

The BALF was processed by centrifuging it at 132×g for 5 mins at 4° C. The resultant supernatant was collected and stored at −80° C. for further assessment. The cell pellet was resuspended with red blood cell (RBC) lysis buffer (0.15M ammonium chloride (Sigma Aldrich, Castle Hill, NSW, Australia), 0.01M sodium bicarbonate (Sigma Aldrich, Castle Hill, NSW, Australia), 0.001M EDTA (Sigma Aldrich, Castle Hill, NSW, Australia) for 5 min at 4° C. 1 ml of PBS was added to stop the activity of the red blood cell lysis buffer. The solution was centrifuged at 132×g for 5 mins at 4° C.; this time the resultant supernatant was discarded and the cell pellet was resuspended with 160 μl of PBS.

The total leukocyte count was calculated using the trypan blue exclusion method using a haemocytometer. The remaining sample was cytocentrifuged at 300 rpm for 10 mins onto clean microscope slides using the cytocentrifuge (Shandon, Cheshire, England). The slides were allowed to dry overnight before being stained with May-Grunwald Giesma stain to allow for enumeration of individual cell types (Thorburn et al., Thorax, 2010, 65(12):1053-60). The enumeration of the inflammatory cells was conducted by counting a total of 200 cells under a light microscope (40× magnification) and the cells were differentiated based on their morphology.

### Lung Processing for Histology

The left lung was perfused by gently injecting 0.9% sodium chloride solution through the apex of the right ventricle of the heart at constant pressure until the lung was inflated and changed its colour to white/pink. The left lung was then inflated and fixed with 10% neutral buffered formalin (Lonza Australia Proprietary Limited, Waverley Vic, Australia) via the trachea. The left lung was then excised and stored in formalin for a minimum of 24 hours to allow the tissue fixation. The fixed-left lung was then transferred to 10% ethanol (AnalaR NORMAPUR® ACS, Avantor) in Phosphate-buffered saline (PBS) (ThermoFisher Scientific, Grand Island, New York).

The lungs were processed using a Leica HistoCore PEARL tissue processor and the processed lungs were paraffin embedded and sectioned using a Leica RM2245 semi-automated microtome. The sections (3.5 m thickness) were mounted on microscope slides. Masson's trichrome staining was used to measure collagen deposition and H&E staining for assessment of emphysema-like alveolar enlargement (Donovan et al., J Leukocyte Biology, 2019, 105(1):143-150).

### Results

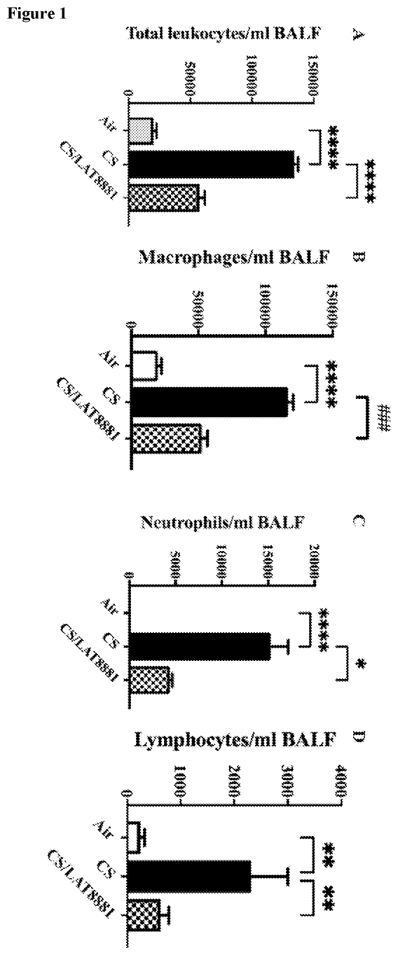

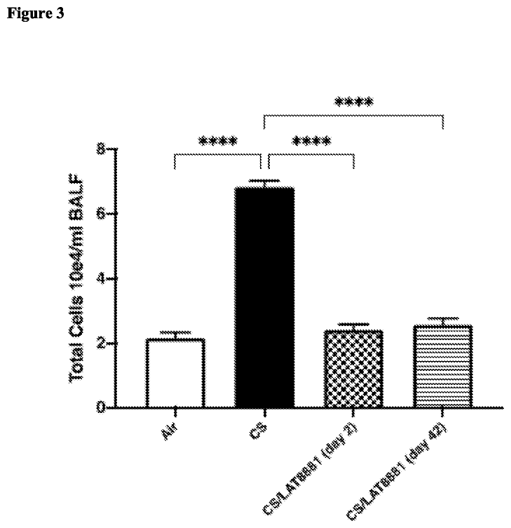

Chronic airway inflammation, which is one of the hallmark features of COPD, was induced by exposing female c57BL/6 mice to cigarette smoke for 2 or 8 weeks. Airway inflammation was assessed by differential counting of inflammatory cells, macrophages, neutrophils and lymphocytes in the bronchoalveolar lavage fluid. The increase in airway inflammation was evident by the increase in the number of total leukocytes, macrophages, neutrophils, lymphocytes and eosinophils in cigarette smoke-exposed mice, as compared to air-exposed mice (Control group; Air) (see FIGS. 1 A-D and FIG. 3). Treatment with LAT8881 showed a significant reduction in airway inflammation in the cigarette smoke-exposed mice, as demonstrated by a decrease in the number of total leukocytes, macrophages, neutrophils and eosinophils (see FIGS. 1 A-D and FIG. 3). Advantageously, treatment with LAT8881 also reversed airway inflammation, as demonstrated by a reduction in the number of total leukocytes in animals that were treated with LAT8881 from day 42 following daily exposure to cigarette smoke.

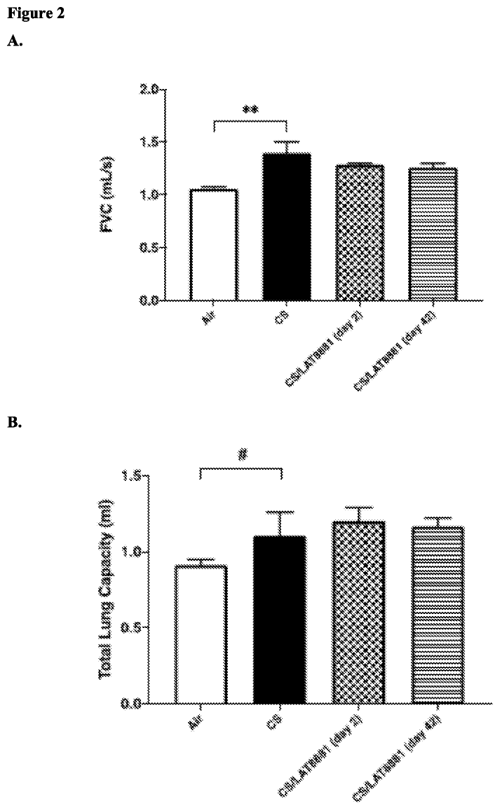

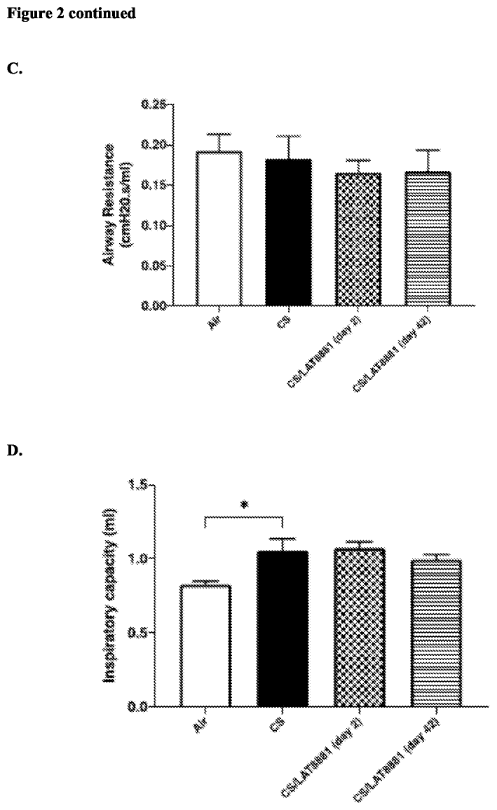

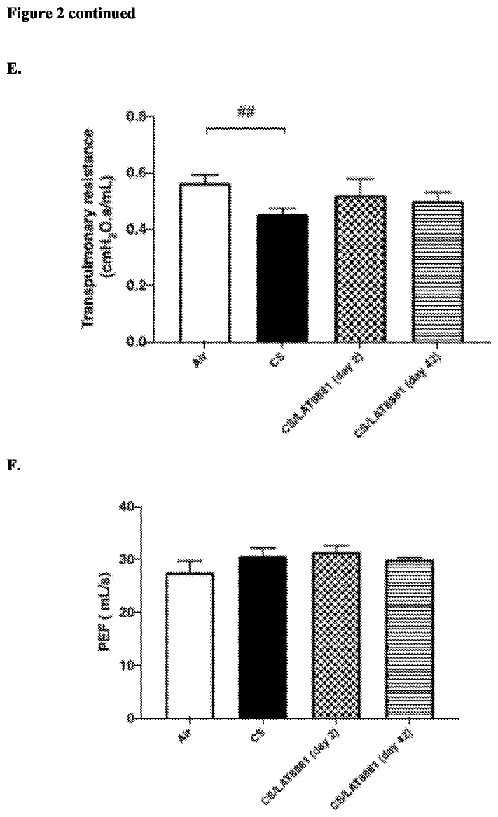

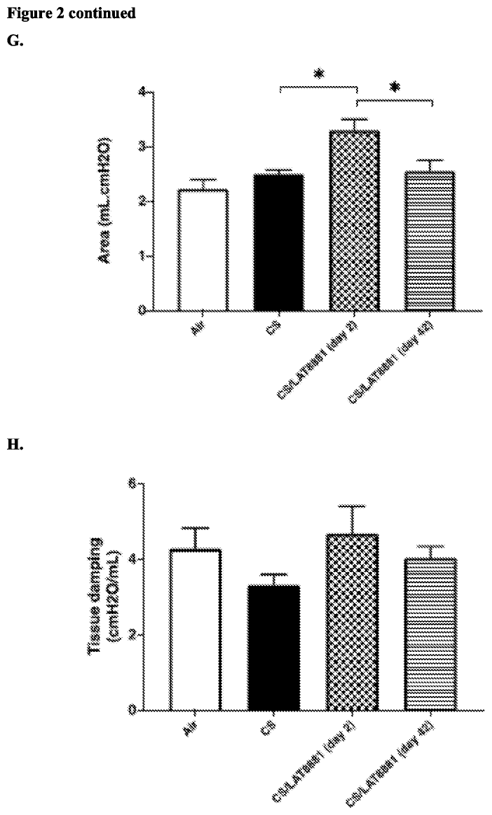

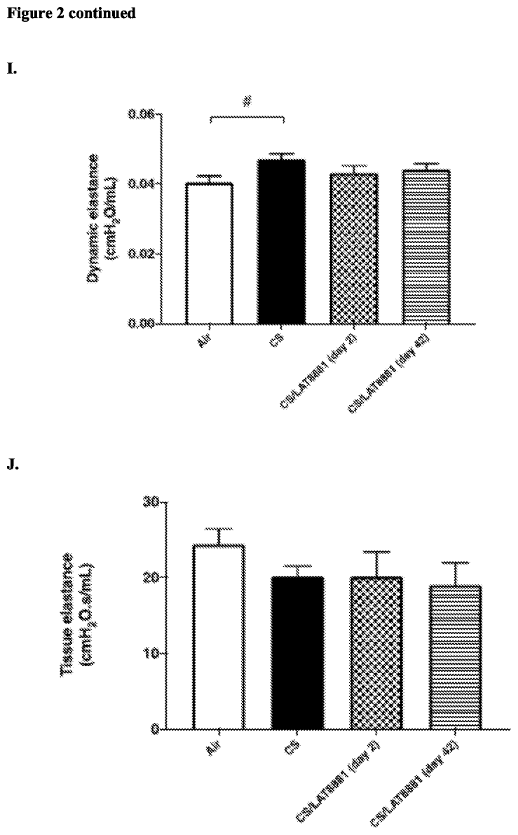

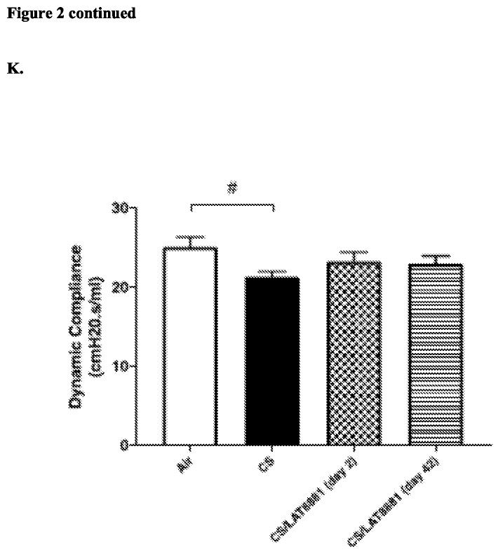

As shown in FIG. 2G, treatment with LAT8881 significantly improved lung compliance when compared to un-treated animals, including those exposed to cigarette smoke.

### Example 3: Effect of Peptides on Airway Responsiveness in an Animal Model of Allergic Airways Disease (AAD)

As previously described by Essilfie et al. (2011, PLoS Pathog; 7:e1002244), 6-8 week old female BALB/c mice were intraperitoneally (IP) sensitised on day 0 to ovalbumin (Ova, 50 g; Sigma-Aldrich, Castle Hill, Australia)), with adjuvant (alum; 26 μl Alhydrogel 2%, Invivogen in 200 μL 0.9% saline). Mice were then intranasally (IN) challenged with Ova on days 12-13 and days 33-34 (20 μg; 50 μL sterile saline).

Mice were subsequently challenged with Chlamydia muridarum on day 14, as follows: For challenge with the natural mouse pathogen Chlamydia muridarum (Cmu), mice were inoculated intranasally with Cmu (100 inclusion-forming units, ATCCVR-123, L sucrose phosphate glutamate buffer (SPG)); and

In the challenge experiments:
(i) Dexamethasone (DEX) was administered IN (2 mg/kg; 50 L phosphate buffered saline (PBS)) on days 32-34 with Ova challenges (Group 6).
(ii) Group 7 were administered 20 mg/kg of LAT8881 (suspended in phosphate-buffered saline) IN in 25 μl;
(iii) Group 8 received both DEX and LAT8881 treatments on days 32-34; and
(iv) Controls were sham sensitised with saline, and sham-inoculated.

Mice were sacrificed 24 hours after the final challenge and airway hyperresponsiveness (AHR) was assessed.

AHR was measured on anaesthetised, cannulated mice using the Scireq flexiVent FX1 system (Montreal, Canada) for all experiments. Briefly, once surgical anaesthesia has been established, a 18 G or 19 G cannula was inserted by tracheostomy and secured to the trachea with a cotton suture. Cannulated mice were connected to the Flexivent apparatus and ventilated with a tidal volume of 8 mL/kg at a rate of 450 breaths per minute. Once stabilised, baseline lung function measurements were collected. Mice were then challenged with aerosolised phosphate buffered saline (PBS), followed by increasing concentrations of acetyl-beta-methylcholine chloride ([methacholine] at 1.25-100 mg/mL). The aerosols were generated using an ultrasonic nebuliser and delivered to the inspiratory line. Each aerosol was delivered for up to 5 minutes during which time regular ventilation was maintained. Upon completion of measurements, mice were euthanased by intraperionteal injection of sodium pentobarbine (>100 mg/kg).

Table 4 (below) provides a summary of the sham/treatment groups in this party of the study.

TABLE 4

| | | | | Number | | |
|---|---|---|---|---|---|---|
| | # mice/ | | | of | | |
| Group | gp | Groups | Treatments | Endpoints | Timepoints | # mice |
| 1 | 8 | Sal/OVA/OVA | N/A | 1 | Day 35 | 8 |
| 2 | 8 | OVA/OVA/OVA | N/A | 1 | Day 35 | 8 |
| 3 | 8 | OVA/OVA/OVA | Dex (d 32-34) | 1 | Day 35 | 8 |
| 4 | 8 | Sal/OVA/OVA + | N/A | 1 | Day 35 | 8 |
| | | NTHi or Cmu | | | | |
| 5 | 8 | OVA/OVA/OVA + | N/A | 1 | Day 35 | 8 |
| | | NTHi or Cmu | | | | |
| 6 | 8 | OVA/OVA/OVA + | Dex (d 32-34) | 1 | Day 35 | 8 |
| | | NTHi or Cmu | | | | |
| 7 | 8 | OVA/OVA/OVA + | 8881 (for 3 days; | 1 | Day 35 | 8 |
| | | NTHi or Cmu | d 32-34) | | | |
| 8 | 8 | OVA/OVA/OVA + | Dex (d 32-34) and | 1 | Day 35 | 8 |
| | | NTHi or Cmu | 8881 (for 3 days; | | | |
| | | | d 32-34) | | | |

### Results

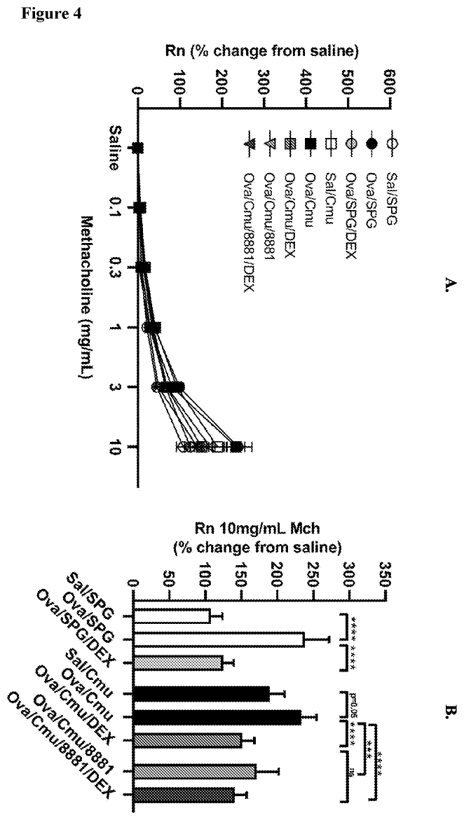

As shown in FIG. 4A, mice challenged with an increasing concentration of methacholine showed increased airway hyperresponsiveness, as evidenced by an increase in airway resistance (Rn). As shown in FIG. 4B, treatment with LAT8881 significantly reduced airway resistance when compared to sham (saline)-treated animals.

1. A method of treating an inflammatory airway disease in a subject, the method comprising administering to a subject in need thereof a therapeutically effective amount of a peptide of formula (I), or a pharmaceutically acceptable salt thereof:
(I)
(SEQ ID NO: 1)
R¹-CRSVEGSCG-R²
wherein
R¹ is selected from the group consisting of YLRIVQ (SEQ ID NO:45), LRIVQ (SEQ ID NO:46), RIVQ (SEQ ID NO:47), IVQ, VQ, and Q, or R¹ is absent; and
R² is F (phenylalanine) or R² is absent.

2. The method of claim 1, wherein the peptide is selected from the group consisting of YLRIVQCRSVEGSCGF (SEQ ID NO:2), LRIVQCRSVEGSCGF (SEQ ID NO:3), CRSVEGSCG (SEQ ID NO:4) and CRSVEGSCGF (SEQ ID NO:5).

3-5. (canceled)

6. The method of claim 1, wherein the subject is a human.

7-18. (canceled)

19. A method of treating an inflammatory airway disease in a subject, the method comprising administering to a subject in need thereof a therapeutically effective amount of a peptide of formula (II), or a pharmaceutically acceptable salt thereof:
(II)
(SEQ ID NO: 6)
R¹-CRRFVESSC-R²
wherein
R¹ is selected from the group consisting of YLRVMK (SEQ ID NO:48), LRVMK (SEQ ID NO:49, RVMK (SEQ ID NO:50), VMK, MK, and K, or R¹ is absent; and
R² is selected from the group consisting of A (alanine) and AF (alanine-phenylalanine), or R² is absent.

20. The method of claim 19, wherein the peptide is selected from the group consisting of YLRVMKCRRFVESSCAF (SEQ ID NO:7), LRVMKCRRFVESSCAF (SEQ ID NO:8), CRRFVESSCAF (SEQ ID NO:9) and CRRFVESSCA (SEQ ID NO:10).

21-23. (canceled)

24. The method of claim 19, wherein the subject is selected from the group consisting of a feline, a canine and an equine.

25-36. (canceled)

37. A method of treating an inflammatory airway disease in a subject, the method comprising administering to a subject in need thereof a therapeutically effective amount of a peptide of formula (III):
(III)
(SEQ ID NO: 11)
R¹-C-R-X¹-X²-P-X³-X⁴-X⁵-X⁶-C-R²
wherein
X¹, X³, X⁵, and X⁶ is an amino acid residue selected from the group consisting of serine, alanine, valine, leucine, isoleucine and glycine;
X² is arginine or lysine;
X⁴ is glutamic acid or aspartic acid;
R¹ is selected from the group consisting of:
S,
(SEQ ID NO: 12)
HS,
(SEQ ID NO: 13)
GHS,
(SEQ ID NO: 14)
PGHS,
(SEQ ID NO: 15)
APGHS,
(SEQ ID NO: 16)
EAPGHS,
(SEQ ID NO: 17)
SEAPGHS,
(SEQ ID NO: 18)
SSEAPGHS,
(SEQ ID NO: 19)
PSSEAPGHS,
(SEQ ID NO: 20)
DPSSEAPGHS
and
(SEQ ID NO: 21)
IDPSSEAPGHS,
or R¹ is absent; and
R² is selected from the group consisting of
S,
(SEQ ID NO: 22)
SS,
(SEQ ID NO: 23)
SSK,
(SEQ ID NO: 24)
SSKF,
(SEQ ID NO: 25)
SSKFS,
(SEQ ID NO: 26)
SSKFSW,
(SEQ ID NO: 27)
SSKFSWD,
(SEQ ID NO: 28)
SSKFSWDE,
(SEQ ID NO: 29)
SSKFSWDEY,
(SEQ ID NO: 30)
SSKFSWDEYE,
(SEQ ID NO: 31)
SSKFSWDEYEQ,
(SEQ ID NO: 32)
SSKFSWDEYEQY,
(SEQ ID NO: 33)
SSKFSWDEYEQYK,
(SEQ ID NO: 34)
SSKFSWDEYEQYKK,
and
(SEQ ID NO: 35)
SSKFSWDEYEQYKKE,
or R² is absent;
or a pharmaceutically acceptable salt thereof.

38. The method of claim 37, wherein the peptide of formula (III) has an amino acid sequence selected from the group consisting of:
(SEQ ID NO: 36)
SCRSRPVESSC;
(SEQ ID NO: 37)
CRSRPVESSC;
(SEQ ID NO: 38)
CRSRPVESSCS;
and
(SEQ ID NO: 39)
SCRSRPVESSCS.

39-42. (canceled)

43. A method of treating an inflammatory airway disease in a subject, the method comprising administering to a subject in need thereof a peptide of formula (IV) or a pharmaceutically acceptable salt thereof:
(IV)
(SEQ ID NO: 40)
R¹-C-R-I-X₁-X₂-X₃-X₄-N-C-R²
wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R₁ is selected from the group consisting of YLKLLK (SEQ ID NO:51), LKLLK (SEQ ID NO:52, KLLK (SEQ ID NO:53, LLK, LK, K or R¹ is absent; and
R² is G (glycine), or R² is absent.

44. The method of claim 43, wherein the peptide of formula (IV) is selected from the group consisting of CRIIHNNNC (SEQ ID NO:41), CRIIHNNNCG (SEQ ID NO:42), CRIVYDSNC (SEQ ID NO:43) and CRIVYDSNCG (SEQ ID NO:44).

45-48. (canceled)

49. The method of claim 1, wherein the inflammatory airway disease is selected from the group consisting of chronic obstructive pulmonary disease (COPD), asthma, chronic bronchitis, emphysema, pulmonary hypertension, cystic fibrosis, lung fibrosis, lung cancer and bronchopulmonary dysplasia.

50. The method of claim 1, wherein the inflammatory airway disease is COPD.

51. (canceled)

52. The method of claim 1, wherein the method comprises administering the peptide to the subject by inhalation or insufflation.

53-59. (canceled)
